SEQUENCE LISTING (35 sequences)

SEQ ID NO: 1 - protein, 609 aa, Escherichia coli
MCGIVGAIAQRDVAEILLEGLRRLEYRGYDSAGLAVVDAEGHMTRLRRLGKVQMLAQAAEEHPLHGGTGIAHTRWATHGEPSEVNAHPHVSEHIVVVHNGIIENHEPLREELKARGYTFVSETDTEVIAHLVNWELKQGGTLREAVLRAIPQLRGAYGTVIMDSRHPDTLLAARSGSPLVIGLGMGENFIASDQLALLPVTRRFIFLEEGDIAEITRRSVNIFDKTGAEVKRQDIESNLQYDAGDKGIYRHYMQKEIYEQPNAIKNTLTGRISHGQVDLSELGPNADELLSKVEHIQILACGTSYNSGMVSRYWFESLAGIPCDVEIASEFRYRKSAVRRNSLMITLSQSGETADTLAGLRLSKELGYLGSLAICNVPGSSLVRESDLALMTNAGTEIGVASTKAFTTQLTVLLMLVAKLSRLKGLDASIEHDIVHGLQALPSRIEQMLSQDKRIEALAEDFSDKHHALFLGRGDQYPIALEGALKLKEISYIHAEAYAAGELKHGPLALIDADMPVIVVAPNNELLEKLKSNIEEVRARGGQLYVFADQDAGFVSSDNMHIIEMPHVEEVIAPIFYTVPLQLLAYHVALIKGTDVDQPRNLAKSVTVE

SEQ ID NO: 2 - protein, 456 aa, Escherichia coli
MLNNAMSVVILAAGKGTRMYSDLPKVLHTLAGKAMVQHVIDAANELGAAHVHLVYGHGGDLLKQALKDDNLNWVLQAEQLGTGHAMQQAAPFFADDEDILMLYGDVPLISVETLQRLRDAKPQGGIGLLTVKLDDPTGYGRITRENGKVTGIVEHKDATDEQRQIQEINTGILIANGADMKRWLAKLTNNNAQGEYYITDIIALAYQEGREIVAVHPQRLSEVEGVNNRLQLSRLERVYQSEQAEKLLLAGVMLRDPARFDLRGTLTHGRDVEIDTNVIIEGNVTLGHRVKIGTGCVIKNSVIGDDCEISPYTVVEDANLAAACTIGPFARLRPGAELLEGAHVGNFVEMKKARLGKGSKAGHLTYLGDAEIGDNVNIGAGTITCNYDGANKFKTIIGDDVFVGSDTQLVAPVTVGKGATIAAGTTVTRNVGENALAISRVPQTQKEGWRRPVKKK

SEQ ID NO: 3 - protein, 609 aa, Escherichia coli; identical to SEQ ID NO: 1

SEQ ID NO: 4 - protein, 284 aa, Escherichia coli
MVLMIVSGRSGSGKSVALRALEDMGFYCVDNLPVVLLPDLARTLADREISAAVSIDVRNMPESPEIFEQAMSNLPDAFSPQLLFLDADRNTLIRRYSDTRRLHPLSSKNLSLESAIDKESDLLEPLRSRADLIVDTSEMSVHELAEMLRTRLLGKRERELTMVFESFGFKHGIPIDADYVFDVRFLPNPHWDPKLRPMTGLDKPVAAFLDRHTEVHNFIYQTRSYLELWLPMLETNNRSYLTVAIGCTGGKHRSVYIAEQLADYFRSRGKNVQSRHRTLEKRKP

SEQ ID NO: 5 - DNA, 855 bp, Escherichia coli
  source 1..855: mol_type = genomic DNA
atggtactgatgatcgtcagcggacgttcaggttcaggtaaatctgtcgccctgcgtgcgctggaagatatgggtttttactgcgtggataaccttcccgtagtgttgttacccgatctggctcgaaccctggccgatcgcgagatttctgccgccgtcagcattgatgttcgtaatatgccggagtcaccagaaatattcgaacaggcgatgagtaacctgcctgacgctttctcaccgcaactcctgttcctggatgccgaccgtaataccttaattcgtcgttacagtgacacgcgccgactgcatccgctttccagcaaaaacctgtcgctggaaagtgctatcgacaaagaaagcgatttgctggagcctctgcgttcgcgagcggatctgattgtcgatacctcagaaatgtccgttcacgagctggctgaaatgctgcgtacccgtctgctgggtaaacgtgaacgcgaactgaccatggtctttgagtctttcggcttcaaacacggtatccctatcgatgcagattacgtctttgacgtgcgcttcttgccgaacccgcactgggatccgaaactgcgtccaatgacaggtcttgataaacctgtcgccgcgttcctcgaccgccacacagaagtacacaattttatctaccagacgcgaagctatcttgagctatggttacctatgctggaaaccaacaaccgtagctacttgacggtcgccattggttgtaccggcgggaagcaccgttcggtgtatattgcagagcaactggcagactacttccgctcgcgcggtaaaaacgtccagtcacgccatcgtacgctggaaaaacgtaaaccatga

SEQ ID NO: 6 - protein, 332 aa, Neisseria meningitidis
MQPLVSVLICAYNVEKYFAQSLAAVVNQTWRNLEILIVDDGSTDGTLAIAKDFQKRDSRIKILAQAQNSGLIPSLNIGLDELAKSGMGEYIARTDADDIAAPDWIEKIVGEMEKDRSIIAMGAWLEVLSEEKDGNRLARHHRHGKIWKKPTRPEDIADFFPFGNPIHNNTMIMRRSVIDGGLRYNTERDWAEDYQFWYDVSKLGRLAYYPEALVKYRLHANQVSSKYSIRQHEIAQGIQKTARNDFLQSMGFKTRFDSLEYRQIKAVAYELLEKHLPEEDFERARRFLYQCFKRTDTLPAGAWLDFAADGRMRRLFTLRQYFGILHRLLKNR

SEQ ID NO: 7 - protein, 439 aa, Helicobacter pylori
MISVYIISLKESQRRLDTEKLVLESNEKFKGRCVFQIFDAISPKHEDFEKFVQELYDSSSLLKSDWFHSDYCYQELLPQEFGCYLSHYLLWKECVKLNQPVVILEDDVALESNFMQALEDCLKSPFDFVRLYGHYWGGHKTNLCALPVYTETEEAEASIEKTPIENYEVTSPPPPNPTRDTQQDFITETQQDPKELSEPCKIAPQKISFNQVVFKKIKRKLNRFIGSILARTEVYKNIVAKYDDLTTKYDDLTTKYDDLTTKYDDLTTKYDDLNKNIAEKYDELMGKYESLLAKEVNIKETFWESRADSEKEALFLDHFYLTSVYVATTAGYYLTPKGAKTFIEATERFKIIEPVDMFINNPTYHDIANFTYVPCPVSLNKHAFNSTIQNAKKPDISLKPPKKSYFDNLFYHKFNARKCLKAFNKYSKQYAPLKTPKEV

SEQ ID NO: 8 - protein, 273 aa, Helicobacter pylori
MRVFAISLNQKVCDTFGLVFRDTTTLLNSINATHHQAQIFDAIYSKTFEGGLHPLVKKHLHPYFITQNIKDMGITTNLISEVSKFYYALKYHAKFMSLGELGCYASHYSLWEKCIELNEAICILEDDITLKEDFKEGLDFLEKHIQELGYIRLMHLLYDASVKSEPLSHKNHEIQERVGIIKAYSEGVGTQGYVITPKIAKVFLKCSRKWVVPVDTIMDATFIHGVKNLVLQPFVIADDEQISTIARKEEPYSPKIALMRELHFKYLKYWQFV

SEQ ID NO: 9 - protein, 303 aa, Pasteurella oralis
MDEVERSRECKSVVVAGNGASLTQVDYSLLPVNYDVFRCNQFYFEDHYFLGKKIKAAFFTPGVLLEQYYTLYHLRKNNEYSVDKIILSSFNHPTVDLSKSENIKKLFIDVINGYEEYLSKLSDFDIYLRYKELYESQRITSGIYMCAVAIAMGYTDIYLTGIDFYESNSKSYAFEPKKTNIISLLPDFKNISSKFHYHNKDADLEALYFLQKNYSVNFYSISPGSLLYEHFPTSTKNISSQTNLIPVKKENYINDILLPPDFVYEKLGFVNSKKERLNANLIFRIIKDFIKLPSAIKHYLREK

SEQ ID NO: 10 - DNA, 203 bp, synthetic construct
  source 1..203: mol_type = other DNA
  misc_feature 1..203: note = promoter
tgcgtcgccattctgtcgcaacacgccagaatgcggcggcgatcactaactcaacaaatcaggcgatgtaaccgctttcaatctgtgagtgatttcacagtatcttaacaatgtgatagctatgattgcaccgtgcctacaagcatcgtggaggtccgtgactttcacgcatacaacaaacattaaccaagagaaaaacagct

SEQ ID NO: 11 - DNA, 203 bp, synthetic construct
  source 1..203: mol_type = other DNA
  misc_feature 1..203: note = Promoter
tgcgtcgccattctgtcgcaacacgccagaatgcggcggcgatcactaactcaacaaatcaggcgatgtaaccgctttcaatctgtgagtgatttcacagtatcttaacaatgtgatagctatgattgcaccgtgcctacaagcatcgtggaggtccgtgactttcacgcatacaacaaacattaaccaactgagaaacagct

SEQ ID NO: 12 - DNA, 203 bp, synthetic construct
  source 1..203: mol_type = other DNA
  misc_feature 1..203: note = Promoter
tgcgtcgccattctgtcgcaacacgccagaatgcggcggcgatcactaactcaacaaatcaggcgatgtaaccgctttcaatctgtgagtgatttcacagtatcttaacaatgtgatagctatgattgcaccgtgcctacaagcatcgtggaggtccgtgactttcacgcatacaacaaacattaacaaaaaccggagatacc

SEQ ID NO: 13 - DNA, 141 bp, synthetic construct
  source 1..141: mol_type = other DNA
  misc_feature 1..141: note = Promoter
tgtgagttagctcactcattaggcaccccaggctttacactttatgcttccggctcgtatgttgtgtggaatgcctacaagcatcgtggaggtccgtgactttcacgcatacaacaaacattaaccaaggaggaaacagct

SEQ ID NO: 14 - DNA, 203 bp, synthetic construct
  source 1..203: mol_type = other DNA
  misc_feature 1..203: note = Promoter
tgcgtcgccattctgtcgcaacacgccagaatgcggcggcgatcactaactcaacaaatcaggcgatgtaaccgctttcaatctgtgagtgatttcacagtatcttaacaatgtgatagctatgattgcaccgtgcctacaagcatcgtggaggtccgtgactttcacgcatacaacaaacattaaccaaaggaaaaacagct

SEQ ID NO: 15 - DNA, 203 bp, synthetic construct
  source 1..203: mol_type = other DNA
  misc_feature 1..203: note = Promoter
tgcgtcgccattctgtcgcaacacgccagaatgcggcggcgatcactaactcaacaaatcaggcgatgtaaccgctttcaatctgtgagtgatttcacagtatcttaacaatgtgatagctatgattgcaccgtgcctacaagcatcgtggaggtccgtgactttcacgcatacaacaaacattaaccaactaggaaacagct

SEQ ID NO: 16 - DNA, 203 bp, synthetic construct
  source 1..203: mol_type = other DNA
  misc_feature 1..203: note = Promoter
tgcgtcgccattctgtcgcaacacgccagaatgcggcggcgatcactaactcaacaaatcaggcgatgtaaccgctttcaatctgtgagtgatttcacagtatcttaacaatgtgatagctatgattgcaccgtgcctacaagcatcgtggaggtccgtgactttcacgcatacaacaaacattaaccaaccgagaaacagct

SEQ ID NO: 17 - DNA, 310 bp, synthetic construct
  source 1..310: mol_type = other DNA
  misc_feature 1..310: note = Promoter
atgcgcaaatgcggcacgccttgcagattacggtttgccacacttttcatccttctcctggtgacataatccacatcaatcgaaaatgttaataaatttgttgcgcgaatgatctaacaaacatgcatcatgtacaatcagatggaataaatggcgcgataacgctcattttatgacgaggcacacacattttaagttcgatatttctcgtttttgctcgttaacgataagtttacagcatgcctacaagcatcgtggaggtccgtgactttcacgcatacaacaaacattaaccaactaggaaacagct

SEQ ID NO: 18 - DNA, 203 bp, synthetic construct
  source 1..203: mol_type = other DNA
  misc_feature 1..203: note = Promoter
tgcgtcgccattctgtcgcaacacgccagaatgcggcggcgatcactaactcaacaaatcaggcgatgtaaccgctttcaatctgtgagtgatttcacagtatcttaacaatgtgatagctatgattgcaccgtgcctacaagcatcgtggaggtccgtgactttcacgcatacaacaaacattaaccaagagcaaaacagct

SEQ ID NO: 19 - DNA, 203 bp, synthetic construct
  source 1..203: mol_type = other DNA
  misc_feature 1..203: note = Promoter
tgcgtcgccattctgtcgcaacacgccagaatgcggcggcgatcactaactcaacaaatcaggcgatgtaaccgctttcaatctgtgagtgatttcacagtatcttaacaatgtgatagctatgattgcaccgtgcctacaagcatcgtggaggtccgtgactttcacgcatacaacaaacattaaccaaggaggaaacagct

SEQ ID NO: 20 - DNA, 189 bp, synthetic construct
  source 1..189: mol_type = other DNA
  misc_feature 1..189: note = Promoter
gaaaacattcataaattaaatgtgaattgccgcacacattattaaataagatttacaaaatgttcaaaatgacgcatgaaatcacgtttcactttcgaattatgagcgaatatgcgcgatgcctacaagcatcgtggaggtccgtgactttcacgcatacaacaaacattaaccaaggaggaaacagct

SEQ ID NO: 21 - DNA, 239 bp, synthetic construct
  source 1..239: mol_type = other DNA
  misc_feature 1..239: note = Promoter
ccatttagccatagtaaaaacatgaattgtttgatttcgcgcatattcgctcataattcgaaagtgaaacgtgatttcatgcgtcattttgaacattttgtaaatcttatttaataatgtgtgcggcaattcacatttaatttatgaatgttttcttaacatcgcggcatgcctacaagcatcgtggaggtccgtgactttcacgcatacaacaaacattaaccaaggaggaaacagct

SEQ ID NO: 22 - DNA, 291 bp, synthetic construct
  source 1..291: mol_type = other DNA
  misc_feature 1..291: note = Promoter
cggcaacctatgcctgatgcgacgctgaagcgtcttatcatgcctacatagcactgccacgtatgtttacaccgcatccggcataaaaacacgcgcactttgctacggcttccctatcgggaggccgtttttttgcctttcactcctcgaataattttcatattgtcgtttttgtgatcgttatctcgatatttaaaaacaaataatttcattatattttgtgcctacaagcatcgtggaggtccgtgactttcacgcatacaacaaacattaaccaaggaggaaacagct

SEQ ID NO: 23 - DNA, 300 bp, synthetic construct
  source 1..300: mol_type = other DNA
  misc_feature 1..300: note = Promoter
gcggcacgccttgcagattacggtttgccacacttttcatccttctcctggtgacataatccacatcaatcgaaaatgttaataaatttgttgcgcgaatgatctaacaaacatgcatcatgtacaatcagatggaataaatggcgcgataacgctcattttatgacgaggcacacacattttaagttcgatatttctcgtttttgctcgttaacgataagtttacagcatgcctacaagcatcgtggaggtccgtgactttcacgcatacaacaaacattaaccaaggaggaaacagct

SEQ ID NO: 24 - DNA, 310 bp, synthetic construct
  source 1..310: mol_type = other DNA
  misc_feature 1..310: note = Promoter
atgcgcaaatgcggcacgccttgcagattacggtttgccacacttttcatccttctcctggtgacataatccacatcaatcgaaaatgttaataaatttgttgcgcgaatgatctaacaaacatgcatcatgtacaatcagatggaataaatggcgcgataacgctcattttatgacgaggcacacacattttaagttcgatatttctcgtttttgctcgttaacgataagtttacagcatgcctacaagcatcgtggaggtccgtgactttcacgcatacaacaaacattaaccaactgagaaacagct

SEQ ID NO: 25 - DNA, 310 bp, synthetic construct
  source 1..310: mol_type = other DNA
  misc_feature 1..310: note = Promoter
atgcgcaaatgcggcacgccttgcagattacggtttgccacacttttcatccttctcctggtgacataatccacatcaatcgaaaatgttaataaatttgttgcgcgaatgatctaacaaacatgcatcatgtacaatcagatggaataaatggcgcgataacgctcattttatgacgaggcacacacattttaagttcgatatttctcgtttttgctcgttaacgataagtttacagcatgcctacaagcatcgtggaggtccgtgactttcacgcatacaacaaacattaaccaaccgagaaacagct

SEQ ID NO: 26 - DNA, 310 bp, synthetic construct
  source 1..310: mol_type = other DNA
  misc_feature 1..310: note = Promoter
atgcgcaaatgcggcacgccttgcagattacggtttgccacacttttcatccttctcctggtgacataatccacatcaatcgaaaatgttaataaatttgttgcgcgaatgatctaacaaacatgcatcatgtacaatcagatggaataaatggcgcgataacgctcattttatgacgaggcacacacattttaagttcgatatttctcgtttttgctcgttaacgataagtttacagcatgcctacaagcatcgtggaggtccgtgactttcacgcatacaacaaacattaaccaagagaaaaacagct

SEQ ID NO: 27 - DNA, 350 bp, synthetic construct
  source 1..350: mol_type = other DNA
  misc_feature 1..350: note = Promoter
tgcgtcgccattctgtcgcaacacgccagaatgcggcggcgatcactaactcaacaaatcaggcgatgtaaccgctttcaatctgtgagtgatttcacagtatcttaacaatgtgatagctatgattgcaccgttttaacgttgtaacccgtatgtaacagtgaataatcacttttgccgaggtaacagcgtcataacaacaattaaagccgttttctggagcgttaccgggcatggaagaacgaattttaaaaagtgagcttcggcgttcagtaacacttcattaactctactgccccgccgagcatttatctcaagcactaccctgcataagcaaggaggaaacagct

SEQ ID NO: 28 - DNA, 310 bp, synthetic construct
  source 1..310: mol_type = other DNA
  misc_feature 1..310: note = promoter
atgcgcaaatgcggcacgccttgcagattacggtttgccacacttttcatccttctcctggtgacataatccacatcaatcgaaaatgttaataaatttgttgcgcgaatgatctaacaaacatgcatcatgtacaatcagatggaataaatggcgcgataacgctcattttatgacgaggcacacacattttaagttcgatatttctcgtttttgctcgttaacgataagtttacagcatgcctacaagcatcgtggaggtccgtgactttcacgcatacaacaaacattaaccaaaggaaaaacagct

SEQ ID NO: 29 - DNA, 300 bp, synthetic construct
  source 1..300: mol_type = other DNA
  misc_feature 1..300: note = Promoter
gcggcacgccttgcagattacggtttgccacacttttcatccttctcctggtgacataatccacatcaatcgaaaatgttaataaatttgttgcgcgaatgatctaacaaacatgcatcatgtacaatcagatggaataaatggcgcgataacgctcattttatgacgaggcacacacattttaagttcgatatttctcgtttttgctcgttaacgataagtttacagcatgcctacaagcatcgtggaggtccgtgactttcacgcatacaacaaacattaaccaagagcaaaacagct

SEQ ID NO: 30 - DNA, 310 bp, synthetic construct
  source 1..310: mol_type = other DNA
  misc_feature 1..310: note = Promoter
atgcgcaaatgcggcacgccttgcagattacggtttgccacacttttcatccttctcctggtgacataatccacatcaatcgaaaatgttaataaatttgttgcgcgaatgatctaacaaacatgcatcatgtacaatcagatggaataaatggcgcgataacgctcattttatgacgaggcacacacattttaagttcgatatttctcgtttttgctcgttaacgataagtttacagcatgcctacaagcatcgtggaggtccgtgactttcacgcatacaacaaacattaaccaagagctaaacagct

SEQ ID NO: 31 - DNA, 189 bp, synthetic construct; identical to SEQ ID NO: 20
  source 1..189: mol_type = other DNA
  misc_feature 1..189: note = Promoter

SEQ ID NO: 32 - DNA, 107 bp, Escherichia coli
  source 1..107: mol_type = genomic DNA
tgtgagttagctcactcattaggcaccccaggctttacactttatgcttccggctcgtatgttgtgtggaattgtgagcggataacaatttcacacaggaaacagct

SEQ ID NO: 33 - DNA, 300 bp, synthetic construct
  source 1..300: mol_type = other DNA
  misc_feature 1..300: note = Promoter
gcggcacgccttgcagattacggtttgccacacttttcatccttctcctggtgacataatccacatcaatcgaaaatgttaataaatttgttgcgcgaatgatctaacaaacatgcatcatgtacaatcagatggaataaatggcgcgataacgctcattttatgacgaggcacacacattttaagttcgatatttctcgtttttgctcgttaacgataagtttacagcatgcctacaagcatcgtggaggtccgtgactttcacgcatacaacaaacattaaccaagaacaaaacagct

SEQ ID NO: 34 - DNA, 300 bp, synthetic construct
  source 1..300: mol_type = other DNA
  misc_feature 1..300: note = Promoter
gcggcacgccttgcagattacggtttgccacacttttcatccttctcctggtgacataatccacatcaatcgaaaatgttaataaatttgttgcgcgaatgatctaacaaacatgcatcatgtacaatcagatggaataaatggcgcgataacgctcattttatgacgaggcacacacattttaagttcgatatttctcgtttttgctcgttaacgataagtttacagcatgcctacaagcatcgtggaggtccgtgactttcacgcatacaacaaacattaaccaaattcgaaacagct

SEQ ID NO: 35 - DNA, 2818 bp, Campylobacter jejuni
  source 1..2818: mol_type = genomic DNA
atgaaagaaataaaaatacaaaatataatcataagtgaagaaaaagcacccttagtcgtacctgaaataggcattaatcataatggcagtttagaactagctaaaattatggtagatgcagcctttagcgcaggtgctaagattataaagcatcaaactcatattgttgaagatgagatgagtaaggccgctaaaaaagtaattcctggtaatgcaaaaataagcatttatgagattatgcaaaaatgtgctttggattataaagatgagctagcacttaaagaatacacagaaaaattaggtcttgtttatcttagcacacctttttctcgtgcaggtgcgaaccgcttagaagatatgggagttagtgcttttaagattggttcaggtgagtgtaataattatccgcttattaaacacatagcagcctttaaaaagcctatgatagttagcacaggaatgaatagtattgaaagtataaaaccaactgtaaaaatcttattagacaatgaaattccttttgttttaatgcacacgaccaatctttacccaaccccgcataatcttgtaagattaaacgctatgcttgagttaaaaaaagaattttcttgtatggtaggcttaagcgaccacacaacagataatcttgcgtgtttaggtgcagttgtacttggagcttgtgtgcttgaaagacattttactgatagtatgcatagaagtggccctgatatagtttgttctatggatacaaaggctttaaaagagctaattatacaaagtgagcaaatggctataataagaggaaataatgaaagtaaaaaagcggctaaacaagaacaagttacaattgattttgcctttgcaagtgtagttagcattaaagatattaaaaaaggcgaagttttatctatggataatatttgggttaaaagacctggacttggtggaattagtgcggctgaatttgaaaatattttaggcaaaaaagcattaagagatatagaaaatgatgctcagttaagctatgaggattttgcgtgaaaaaaatcctttttataacaggctctagggctgattattctaagattaaatctttaatgtacagggtgcaaaactcaagcgaatttgaactttacatctttgcaacaggaatgcacttaagtaaaaattttggctatacagttaaagaactttataaaaatggctttaaaaatatttatgaatttataaattatgataaatattatcaaactgataaggctttagctactacaattgatggattttcaaggtatgcaaatgagctaaaacctgatttaatcgtagtacatggagatagaattgagcctttagcagcagctattgttggagcattaaataatatcttagtagcgcatattgaaggcggagagatttcaggaactattgacgatagcttacgccacgctatatcaaaactagctcatattcatttagtaaatgatgagtttgcaaaaaggcgtttaatgcagcttggagaagatgaaaaatctatttttatcataggttcgcctgatttagaacttttaaacgataataaaatttcacttagcgaagcaaaaaaatattatgatataaattatgaaaactacgctttgcttatgtttcatcctgttacaactgaaattactagcattaaaaatcaagcagacaatttagtaaaagcactgatacaaagtaataaaaattatattgttatttatccaaataatgatttaggttttgaattaatcttgcaaagctatgaagagtttaaaaataaccctagatttaagctttttccatcgcttagatttgagtattttataactttgttaaaaaatgctgattttataataggtaattcaagttgtattttaaaagaggccttatacttaaaaacagcagggattttagttggctcaagacaaaatggaagacttggcaatgaaaatacactaaaagttaatgcaaatagtgatgaaatactaaaagctattaacactattcataaaaaacaagatttatttagcgctaagttagagattttagatagctcaaaattattttttgaatatttacaaagcggagatttttttaaactcagcacacaaaaagtttttaaggatataaaatgagcttagcaataatccctgctcgtggtggctcaaagggtattaaaaataaaaatttggttttattaaacaataaacctttaatttactacacgatcaaagctgcactaaatgctaaaagcattagtaaagttgttgtaagcagtgatagtgatgaaattttaaattatgcaaaaagtcaaaatgttgatattttaaaacgcccaattagccttgcacaagatgataccacaagcgataaagtgctgttacatgctctaaaattttataaagattatgaagatgtagtttttttacaacccacttcaccgctaagaacaaatattcatattaatgaagcttttaatctttataaaaatagcaatgcaaatgccctaattagcgtaagcgaatgtgataataaaattctaaaagcctttgtttgtaatgattgtggcgatttagcagggatttgtaatgatgaatatccttttatgccaaggcaaaaattgcctaaaacttatatgagcaatggtgcaatttatattttaaagataaaagaatttttaaacaatcctagctttttacaaagcaaaaccaagcattttttaatggacgaaagctcaagtttagatattgactgtttggaggatttaaaaaaggttgaacagatatggaaaaaataa

(19) United States
(12) Patent Application Publication
(10) Pub. No.: US 2026/0201318 A1
(43) Pub. Date: Jul. 16, 2026

(54) GENETICALLY MODIFIED UDP-N-ACETYLGALACTOSAMINE PRODUCING CELLS

(71) Applicant: DSM IP Assets B.V., Maastricht (NL)

(72) Inventor: Manos PAPADAKIS, Hørsholm (DK)

(21) Appl. No.: 19/136,681

(22) PCT Filed: Dec. 8, 2023
(86) PCT No.: PCT/EP2023/084921

§ 371 Date: Jun. 6, 2025

(30) Foreign Application Priority Data
Dec. 9, 2022 (DK) PA202201117

(51) Int. Cl. C12N 1/20; C12N 1/14; C12N 9/10; C12N 9/12; C12N 9/90; C12N 15/10; C12P 19/04; C12P 19/44; C12R 1/01; C12R 1/645

(52) U.S. Cl. CPC C12N 1/20; C12N 1/14; C12N 9/1051; C12N 9/1096; C12N 9/1241; C12N 9/90; C12N 15/102; C12P 19/04; C12P 19/44; C12Y 204/01038; C12Y 206/01016; C12Y 207/07023; C12Y 504/0201; C12R 2001/01; C12R 2001/645

(57) ABSTRACT

The present disclosure relates to a genetically modified cell capable of producing UDP-N-acetylglucosamine (UDP-GlcNAc), wherein the mRNA stability of the native glucosamine-6-phopshate synthase (glmS) is enhanced by modifying said cell such that it has decreased or total loss of function of RNase adapter protein RapZ (yhbJ). In particular, the present disclosure relates to said cells that are likewise capable of producing one or more N-acetylated- and/or sialylated molecules, such as N-acetylated- and/or sialylated human milk oligosaccharides (HMOs). The present disclosure further relates to a method for producing one or more N-acetylated- and/or sialylated human milk oligosaccharides (HMO) making use of such cells.

### FIELD

The present disclosure relates to a genetically modified cell capable of producing UDP-N-acetylglucosamine (UDP-GlcNAc), wherein the mRNA stability of the native glucosamine-6-phopshate synthase (glmS) is enhanced by modifying said cell such that it has decreased or total loss of function of RNase adapter protein RapZ (yhbJ). In particular, the present disclosure relates to said cells that are further modified to produce one or more N-acetylated- and/or sialylated molecules, such as N-acetylated- and/or sialylated human milk oligosaccharides (HMOs). The present disclosure further relates to a method for producing one or more N-acetylated- and/or sialylated molecules, such as human milk oligosaccharides (HMO) making use of such cells.

### BACKGROUND

Uridine diphosphate-N-acetylglucosamine (UDP-GlcNAc) is an acetylated amino sugar nucleotide that naturally serves as precursor in bacterial cell wall synthesis and is involved in prokaryotic and eukaryotic glycosylation reactions. UDP-GlcNAc finds application in various fields including the production of oligosaccharides and glycoproteins with therapeutic benefits.

Physiological levels of UDP-GlcNAc in a cell play an important role in the biotechnological production of various compounds. In particular, the levels of UDP-GlcNAc in a cell may have an influence on the yield of a desired compound during its biotechnological production, as UDP-GlcNAc plays a key role in the biosynthetic pathways of a whole range of desired compounds, in particular in its function as a glycosyl donor.

For instance, the cell-based biotechnological production of human milk oligosaccharides (HMOs) and the HMO yields generated thereby, are known to be influenced by the physiological levels of UDP-GlcNAc, as said compound is involved in the biosynthetic pathway leading to the formation of certain human milk oligosaccharides.

Hence, it would be desirable to provide cells for the biotechnological production of certain human milk oligosaccharides that show high levels of UDP-GlcNAc to increase the yields of HMOs. However, it is challenging to find suitable cell modifications that are practically feasible and that provide for a sufficiently high increase of the UDP-GlcNAc concentration and HMO yield, respectively.

In relation to HMO production, examples of increasing expression of phosphoglucosamine mutase (GlmM), N-acetylglucosamine-1-phosphate uridyltransferase (GlmU), and/or glucosamine-6-phopshate synthase (GlmS) are for example described in WO2014/153253 and Zhu et al 2021 J Agric Food Chem 69:3702-3711.

Sugita and Koketsu describe a slight increase in LNT-II in a cell comprising a yhbJ deletion, there is no disclosure of an increased UDP-GlcNAc pool in the cell (Sugita and Koketsu 2022 J. Agric. Food Chem. 70:5106-5114).

Moreover, during the biotechnological production of human milk oligosaccharides often more than one human milk oligosaccharide is produced by a specific cell. However, in some cases only one or two specific human milk oligosaccharides are in fact desired within a produced mixture of human milk oligosaccharides. Therefore, apart from the mere optimization of the overall HMO yield during a biotechnological production process, it would also be desirable to provide modifications of a cell that allow for the pronounced formation of specific human milk oligosaccharides within a produced mixture of human milk oligosaccharides, i.e. to increase the proportion of certain human milk oligosaccharides within a mixture of human milk oligosaccharides.

### BRIEF DESCRIPTION OF THE FIGURES

FIG. 1. Schematic overview of the metabolic pathway responsible for UDP-GlcNAc biosynthesis. Proteins are indicated in bold.

FIG. 2. Concentrations of UDP-GlcNAc for various modified strains and control strain MDO.

FIG. 3. Shows total and individual HMO concentrations produced by LNnT producing strains M12 and M13 having two copies of a beta-1,3-N-acetyloglucosamine transferase, LgtA gene.

FIG. 3A shows the total and individual HMO concentrations (mM) relative to the total amount of HMO produced by the control strain (MP12). FIG. 3B shows the concentration (mM) of the individual HMO relative to the concentration of the specific HMO in the control strain (MP12).

FIG. 4. Shows total and individual HMO concentrations produced by LNnT producing strains M14 and M15 having one copy of a beta-1,3-N-acetyloglucosamine transferase, LgtA gene.

FIG. 4A shows the total and individual HMO concentrations (mM) relative to the total amount of HMO produced by the control strain (MP14). FIG. 4B shows the concentration (mM) of the individual HMO relative to the concentration of the specific HMO in the control strain (MP14).

FIG. 5. Shows total and individual HMO concentrations produced by LNT producing strains M16 and M17 having two copies of a beta-1,3-N-acetyloglucosamine transferase, LgtA gene.

FIG. 5A shows the total and individual HMO concentrations (mM) relative to the total amount of HMO produced by the control strain (MP16). FIG. 5B shows the concentration (mM) of the individual HMO relative to the concentration of the specific HMO in the control strain (MP16).

FIG. 6. Shows total and individual HMO concentrations produced by LNT producing strain MP18 and M19 having one copy of a beta-1,3-N-acetyloglucosamine transferase, LgtA gene.

FIG. 6A shows the total and individual HMO concentrations (mM) relative to the total amount of HMO produced by the control strain (MP18). FIG. 6B shows the concentration (mM) of the individual HMOs in MP19 relative to the concentration of the specific HMO in the control strain (MP18). The control strain was set a 100% and is not shown.

FIG. 7. Relative concentrations of 3′SL in test strain MP21 (ΔyhbJ), MP22 (ΔyhbJ+glmS), and MP23 (ΔyhbJ+glmU) compared to control strain MP20.

FIG. 8. Relative concentrations of LNT-II in test strain MP25 (ΔyhbJ+glmU+glmM) compared to control strain MP24.

### SUMMARY

The present disclosure relates to a genetically modified cell capable of producing UDP-N-acetylglucosamine (UDP-GlcNAc), wherein the mRNA stability of the native glucosamine-6-phopshate synthase (glmS) is enhanced by modifying said cell such that it has decreased or total loss of function of RNase adapter protein RapZ (yhbJ).

In one embodiment the genetically modified cell is further modified to overexpress phosphoglucosamine mutase (glmM) and/or N-acetylglucosamine-1-phosphate uridyltransferase (glmU), and/or overexpression of glucosamine-6-phopshate synthase (glmS). Preferably, such a cell is capable of producing a N-acetylated- and/or sialylated molecule, such as a N-acetylglucosamine containing oligosaccharide, lipid or protein or a sialylated oligosaccharide, lipid or protein.

In one embodiment the genetically modified cell comprises an yhbJ deletion and expresses an alpha-2,3-sialyltransferase or alpha-2,6-sialyltransferase and a biosynthetic pathway for making a sialic acid sugar nucleotide. Preferably, such a cell is capable of producing a sialylated oligosaccharide.

In a particular embodiment, the genetically modified cell according to the disclosure is capable of producing one or more human milk oligosaccharides (HMOs), in particular N-acetylated- and/or sialylated HMOs.

Another aspect of the present disclosure relates to a method for producing one or more N-acetylated- and/or sialylated molecules comprising the steps of:
a) Providing a genetically modified cell according to the present disclosure that is capable of producing one or more N-acetylated- and/or sialylated molecules; and
b) Culturing the cell according to (a) in a suitable cell culture medium to produce said molecule.

Another aspect of the present disclosure concerns a human milk oligosaccharides (HMO) produced by the method according to the disclosure.

Another aspect of the present disclosure concerns the use of a genetically modified cell according to the present disclosure that is capable of producing one or more human milk oligosaccharides for the production of human milk oligosaccharides (HMO).

### DETAILED DESCRIPTION

### Cells Capable of Producing UDP-N-Acetylglucosamine (UDP-GlcNAc)

The present disclosure relates to a genetically modified cell capable of producing UDP-N-acetylglucosamine (UDP-GlcNAc), wherein the mRNA stability of the native glucosamine-6-phopshate synthase (glmS) is enhanced by modifying said cell such that it has decreased or total loss of function of RNase adapter protein RapZ (yhbJ).

In the present context, the terms “genetically modified cell” and “genetically engineered cell” can be used interchangeably. As used herein “a genetically modified cell” is a host cell whose genetic material has been altered by human intervention using a genetic engineering technique, such a technique is e.g. but not limited to transformation or transfection, e.g. with a heterologous polynucleotide sequence, Crispr/Cas editing and/or random mutagenesis. In one embodiment the genetically engineered cell has been transformed or transfected with a recombinant nucleic acid sequence.

The genetically modified cell according to the disclosure is characterized in that it is capable of producing UDP-N-acetylglucosamine (UDP-GlcNAc).

Hence, the modified cell according to the disclosure has the genetic capabilities to produce UDP-N-acetylglucosamine (UDP-GlcNAc).

In one embodiment, the genetically modified cell is capable of producing UDP-N-acetylglucosamine (UDP-GlcNAc) by a de novo pathway. In this regard, UDP-GlcNAc is made by the cell under the action of enzymes involved in the de novo biosynthetic pathway in a stepwise reaction sequence starting from a simple carbon source like glycerol, sucrose, fructose or glucose.

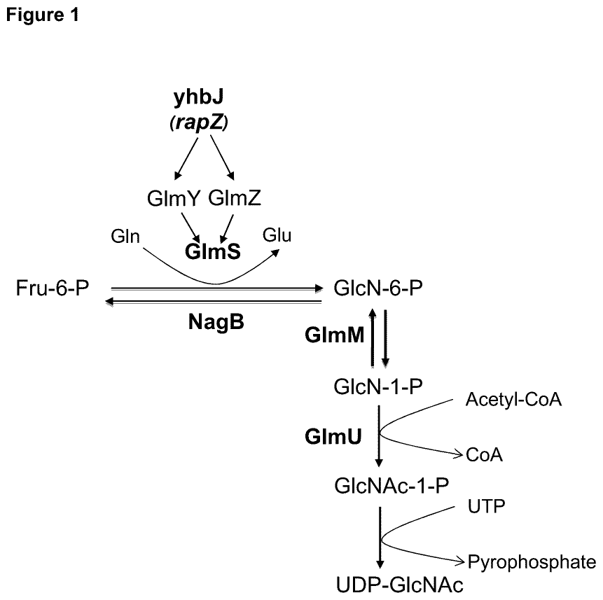

The enzymes involved in the de novo biosynthetic pathway of UDP-GlcNAc can be naturally present in the cell or be introduced into the cell by means of gene technology or recombinant DNA techniques, all of them are parts of the general knowledge of the skilled person. FIG. 1 illustrates the de novo biosynthetic pathway of UDP-GlcNAc.

UDP-GlcNAc is a nucleotide-activated sugar that may serve as a glycosyl donor in various biosynthetic pathways, wherein the glycosylation reaction is mediated by the respective glycosyltransferase.

The genetically modified cell according to the disclosure is further characterized in that the mRNA stability of the native glucosamine-6-phopshate synthase (glmS) is enhanced by modifying said cell such that it has decreased or total loss of function of RNase adapter protein RapZ (yhbJ).

By “native” glucosamine-6-phopshate synthase (glmS), it is meant that the glmS gene naturally occurs in the cell and has not been introduced into the cell via a genetic engineering technique, or otherwise manipulated to increase its expression, e.g., by recombinantly introducing a stronger promoter or other regulatory sequences.

The yhbJ gene encodes the RNase adapter protein RapZ (RapZ) that has an influence on the stability of the messenger RNA encoding the GlmS enzyme, which can be a rate-limiting factor in the biosynthesis of UDP-GlcNAc in cells. The inventors of the present disclosure now found that a cell modification which leads to decreased or total loss of function of RNase adapter protein RapZ is beneficial for increasing the levels of UDP-N-acetylglucosamine (UDP-GlcNAc) in a cell. In E. Coli the ybhJ gene comprises or consists of the nucleic acid sequence according to SEQ ID NO: 5, which encodes the protein RapZ, comprising or consisting of an amino acid sequence according to SEQ ID NO: 4.

The underlying mechanism of the RNase adapter protein RapZ influencing the stability of the messenger RNA encoding the GlmS enzyme is as follows: The two small RNAs glmY and glmZ are involved in the regulation of GlmS expression, in that glmZ stabilizes the glmS mRNA and allows for the expression of GlmS. RapZ (encoded by yhbJ) is able to bind to both glmY and glmZ. When glucosamine-6-phosphate (GlcN-6-P) concentrations are high in a cell, the small RNA glmY is present in low amounts. Under these conditions, RNase adapter protein RapZ is free to bind another small RNA, glmZ, and recruit it to processing by RNase E through protein-protein interaction. Consequently, glmZ is inactivated and thus unable to stabilize the glmS mRNA, leading to GlmS degradation. By contrast, when GlcN-6-P levels decrease, glmY accumulates and binds and sequesters RNase adapter protein RapZ. Thereby, glmZ remains unbound and cannot be processed by RNase E. As a result, glmZ base-pairs with glmS in an Hfq-dependent manner and activates synthesis of GlmS, which resynthesizes GlcN-6-P. In other words, the RNase adapter protein RapZ is involved in the feedback coupling mechanism that controls the synthesis of GlmS.

The inventors of the present disclosure now found that the decreased or total loss of function of RNase adapter protein RapZ leads to the stabilization of the messenger RNA encoding the GlmS enzyme, which in turn leads to an increased synthesis of GlmS enzyme. Since, the glucosamine-6-phopshate synthase (GlmS) is the enzyme that mediates the biosynthesis of GlcN-6-P from Fru-6-P (Fructose-6-phosphate) within the biosynthesis pathway leading to UDP-N-acetylglucosamine (UDP-GlcNAc), the inventors found that thereby the levels of UDP-N-acetylglucosamine (UDP-GlcNAc) can be effectively increased in a cell.

In a preferred embodiment, the decreased or total loss of function of RNase adapter protein RapZ (yhbJ) is achieved by the deletion or by rendering dysfunctional the gene encoding the RNase adapter protein RapZ (yhbJ).

The terms “deletion” and “rendering dysfunctional” refer to the native yhbJ gene and may include 1) partial or complete removal of the gene's coding sequence, 2) the introduction of stop codon(s) in its coding sequence, 3) the inactivation or marked weakening of the gene's promoter or Shine-Dalgarno sequence, 4) the deletion of the gene encoding a direct activator or the enhancement of the expression of a gene encoding a direct repressor of the native promoter of the yhbJ (rapZ) gene, 5) the gene inactivation by insertion of an expression cassette encoding another gene product, such as, but not limited to a drug or antibiotic marker or a glycosyl transferase.

The genetically engineered cell may be any cell capable of producing UDP-N-acetylglucosamine (UDP-GlcNAc). Preferably, the host cell is a unicellular microorganism of eucaryotic or prokaryotic origin. Appropriate microbial cells that may function as a host cell include yeast cells, bacterial cells, archaebacterial cells, algae cells, and fungal cells.

The genetically engineered cell (host cell) may be e.g., a bacterial or yeast cell. In one preferred embodiment, the genetically engineered cell is a bacterial cell.

Regarding the bacterial host cells, there are, in principle, no limitations; they may be eubacteria (gram-positive or gram-negative) or archaebacteria, as long as they allow genetic manipulation for insertion of a gene of interest and can be cultivated on a manufacturing scale. Preferably, the host cell has the property to allow cultivation to high cell densities. Non-limiting examples of suitable bacterial host cells are Escherichia coli, Erwinia herbicola (Pantoea agglomerans), Citrobacter freundii, Campylobacter sp, Corynebacterium sp., Pantoea citrea, Pectobacterium carotovorum, or Xanthomonas campestris. Bacteria of the genus Bacillus may also be used, including Bacillus subtilis, Bacillus licheniformis, Bacillus coagulans, Bacillus thermophilus, Bacillus laterosporus, Bacillus megaterium, Bacillus mycoides, Bacillus pumilus, Bacillus lentus, Bacillus cereus, and Bacillus circulans. Similarly, bacteria of the genera Lactobacillus and Lactococcus may be used, including but not limited to Lactobacillus acidophilus, Lactobacillus salivarius, Lactobacillus plantarum, Lactobacillus helveticus, Lactobacillus delbrueckii, Lactobacillus rhamnosus, Lactobacillus bulgaricus, Lactobacillus crispatus, Lactobacillus gasseri, Lactobacillus casei, Lactobacillus reuteri, Lactobacillus jensenii, and Lactococcus lactis. Streptococcus thermophiles, Streptomyces lividans, and Proprionibacterium freudenreichii are also suitable bacterial species for the present disclosure. Also included as part of this disclosure are strains from the genera Enterococcus (e.g., Enterococcus faecium and Enterococcus thermophiles), Bifidobacterium (e.g., Bifidobacterium longum, Bifidobacterium infantis, and Bifidobacterium bifidum), Sporolactobacillus spp., Micromomospora spp., Micrococcus spp., Rhodococcus spp., and Pseudomonas (e.g., Pseudomonas fluorescens and Pseudomonas aeruginosa).

Non-limiting examples of fungal host cells that are yeast cells, such as Komagataella phaffii, Kluyveromyces lactis, Yarrowia lipolytica, Pichia pastoris, and Saccharomyces cerevisiae or filamentous fungi such as Aspargillus sp, Fusarium sp or Thricoderma sp, exemplary species are A. niger, A. nidulans, A. oryzae, F. solani, F. graminearum and T. reesei.

In one or more exemplary embodiments, the genetically engineered cell is selected from the group consisting of Yarrowia lipolytica, Pichia pastoris, and Saccharomyces cerevisiae.

In one embodiment, the genetically engineered cell is Pichia pastoris.

In one embodiment, the genetically engineered cell is selected from the group consisting of Escherichia sp., Bacillus sp., Lactobacillus sp and Corynebacterium sp. Campylobacter sp.

In one embodiment, the genetically engineered cell is selected from the group consisting of E. coli, C. glutamicum, L. lactis, B. subtilis, S. lividans.

In one embodiment, the genetically engineered cell is Bacillus subtilis.

In one embodiment, the genetically engineered cell is Corynebacterium glutamicum.

In a preferred embodiment, the genetically modified cell is a microbial cell, such as a cell selected from the group consisting of Escherichia coli, Bacillus subtilis, Lactobacillus lactis, Corynebacterium glutamicum, Campylobacter sp., Yarrowia lipolytica, Pichia pastoris, and Saccharomyces cerevisiae.

In a preferred embodiment, the genetically engineered cell is Escherichia coli, preferably E. coli K-12 strain or E. coli DE3, most preferably E. coli K-12.

The UDP-GlcNAc de novo pathway as shown in FIG. 1 comprises three glucosamine modifying enzymes (Glm), starting with glucosamine-6-phopshate synthase (glmS) facilitating the conversion of Fru-6-P to GlcN-6-P. GlcN-6-P is then converted to GlcN-1-P by glucosamine-6-phopshate synthase (GlmS) and finally N-acetylglucosamine-1-phosphate uridyltransferase (GlmU) converts GlcN-1-P to GlcNAc-1-P and then in a second reaction to UDP-GlcNAc. Once removing one limiting factor in the UDP-GlcNAc formation by increasing GlmS through the deletion of the yhbJ gene, possibly a new limiting factor downstream of GlmS may occur, which may be relieved by overexpressing one or more of the Glm enzymes.

In embodiments, the cell further comprises one or more of the following modifications:
a) overexpression of N-acetylglucosamine-1-phosphate uridyltransferase (GlmU), and/or
b) overexpression of glucosamine-6-phopshate synthase (GlmS), and/or
c) overexpression of phosphoglucosamine mutase (GlmM).

Within the biosynthesis pathway of UDP-N-acetylglucosamine (UDP-GlcNAc), the enzyme phosphoglucosamine mutase (GlmM) mediates the formation of GlcN-1-P (glucosamine-1-phosphate) from GlcN-6-P. The inventors of the present disclosure surprisingly found that a cell that shows both, a decreased or total loss of function of RNase adapter protein RapZ (yhbJ) and an overexpression of phosphoglucosamine mutase (GlmM) yields more UDP-N-acetylglucosamine (UDP-GlcNAc) than could have been expected based on the mere additive effects of the aforementioned cell modifications. Insofar, the inventors of the present disclosure found that a synergistic effect occurs between those two modifications.

In a preferred embodiment, the cell is modified further to overexpress phosphoglucosamine mutase (GlmM).

In further embodiments, the cell further comprises at least one of the following modifications:
a) overexpression of N-acetylglucosamine-1-phosphate uridyltransferase (GlmU), and/or
b) overexpression of glucosamine-6-phopshate synthase (GlmS).

Although the level of GlmS is increased through the stabilization of the glmS mRNA through the loss of function of RapZ, it may be beneficial to increase the expression even further by genetically modifying the glmS gene.

In a particular embodiment, the cell comprises the following modifications:
a) deleted or dysfunctional yhbJ gene, and
b) overexpression of glucosamine-6-phopshate synthase (GlmS).

In a particular embodiment, the cell comprises the following modifications:
a) deleted or dysfunctional yhbJ gene, and
b) overexpression of N-acetylglucosamine-1-phosphate uridyltransferase (GlmU).

In a particular embodiment, the cell comprises the following modifications:
a) deleted or dysfunctional yhbJ gene, and
b) overexpression of phosphoglucosamine mutase (GlmM).

In a particular embodiment, the cell comprises the following modifications:
a) deleted or dysfunctional yhbJ gene, and
b) overexpression of N-acetylglucosamine-1-phosphate uridyltransferase (GlmU), and
c) overexpression of glucosamine-6-phopshate synthase (GlmS).

In a particular embodiment, the cell comprises the following modifications:
a) deleted or dysfunctional yhbJ gene, and
b) overexpression of N-acetylglucosamine-1-phosphate uridyltransferase (GlmU), and
c) overexpression of phosphoglucosamine mutase (GlmM).

In a particular embodiment, the cell comprises the following modifications:
a) deleted or dysfunctional yhbJ gene, and
b) overexpression of glucosamine-6-phopshate synthase (GlmS), and
c) overexpression of phosphoglucosamine mutase (GlmM).

In a particular embodiment, the cell comprises the following modifications:
a) deleted or dysfunctional yhbJ gene, and
b) overexpression of N-acetylglucosamine-1-phosphate uridyltransferase (GlmU), and
c) overexpression of glucosamine-6-phopshate synthase (GlmS), and
d) overexpression of phosphoglucosamine mutase (GlmM).

The use of the term “overexpression” e.g., of one or more of the GlmS, GlmM and GlmU proteins, refers to an elevated level of a protein being expressed compared to the normal level of protein expression of a native protein in said cell. It is preferred that the overexpression, in the context of the present disclosure, is achieved directly by manipulation of the target gene. As described herein the level of GlmS can be increased, for example, through the loss of function of the RapZ protein which indirectly serves to stabilize the glmS mRNA. The level of GlmS can further be increased by directly manipulating the expression of the glmS gene, e.g., by modification of the gene copy number, controlling the expression of any copy of a gene at the transcriptional or the translational level, e.g. by substituting the native promoter with a strong promoter, deleting of regulatory elements in the gene that repress the expression of the gene, or introduction of an episomal element, such as a plasmid, that bears and expresses the coding sequence of the gene of interest.

In embodiments the glmS, glmM and/or glmU, gene(s) (target gene) is overexpressed by direct manipulation of the gene, such as i) increasing the promoter strength regulating the expression of the target gene, ii) modification of the native Shine-Dalgarno sequence of the target gene by a stronger sequence with the goal of promoting ribosomal binding, iii) increasing the chromosomal copy number of the target gene, i.e., by inserting one or more additional copies of the target gene in a genomic locus other than the native locus and iv) episomal expression of the target gene from a low (5-10 copies per cell) to a high-copy number plasmid (300-500 copies per cell).

In a particular embodiment, the overexpression is obtained by
a) replacing the native promoter of the gene encoding
   i. phosphoglucosamine mutase (glmM), and/or
   ii. N-acetylglucosamine-1-phosphate uridyltransferase (glmU), and/or
   iii. glucosamine-6-phopshate synthase (glmS),
with a stronger promoter; and/or
b) inserting a recombinant nucleic acid encoding
   i. phosphoglucosamine mutase (glmM) comprising or consisting of the amino acid sequence of SEQ ID NO: 1 or an amino acid sequence with at least 80% sequence identity to SEQ ID NO: 1
   ii. N-acetylglucosamine-1-phosphate uridyltransferase (glmU) comprising or consisting of the amino acid sequence of SEQ ID NO: 2 or an amino acid sequence with at least 80% sequence identity to SEQ ID NO: 2
   iii. glucosamine-6-phopshate synthase comprising or consisting of the amino acid sequence of SEQ ID NO: 3 or an amino acid sequence with at least 80% sequence identity to SEQ ID NO: 3, into said cell.

In the present context, the term “recombinant nucleic acid sequence”, “recombinant gene/nucleic acid/nucleotide sequence/DNA encoding” or “coding nucleic acid sequence” is used interchangeably and intended to mean an artificial nucleic acid sequence (i.e. produced in vitro using standard laboratory methods for making nucleic acid sequences) that comprises a set of consecutive, non-overlapping triplets (codons) which is transcribed into mRNA and translated into a protein when under the control of the appropriate control sequences, i.e. a promoter sequence. A recombinant nucleic acid may be an endogenous gene which has been manipulated, for example, to increase its expression level e.g., by introducing a stronger promoter.

The boundaries of the coding sequence are generally determined by a ribosome binding site located just upstream of the open reading frame at the 5′end of the mRNA, a transcriptional start codon (AUG, GUG or UUG), and a translational stop codon (UAA, UGA or UAG). A coding sequence can include, but is not limited to, genomic DNA, cDNA, synthetic, and recombinant nucleic acid sequences.

The term “nucleic acid” includes RNA, DNA and cDNA molecules. It is understood that, as a result of the degeneracy of the genetic code, a multitude of nucleic acid sequences encoding a given protein may be produced.

The recombinant nucleic acid sequence may be heterologous. As used herein “heterologous” refers to a nucleic acid sequence or nucleotide sequence that is foreign to a cell or organism, i.e. to a nucleic acid sequence that does not naturally occur in said cell.

Increasing the promoter strength driving the expression of the desired target gene may be one way to achieve overexpression. The strength of a promoter can be assed using a lacZ enzyme assay where β-galactosidase activity is assayed as described previously (see e.g. Miller J. H. Experiments in molecular genetics, Cold spring Harbor Laboratory Press, NY, 1972). Briefly the cells are diluted in Z-buffer and permeabilized with sodium dodecyl sulfate (0.1%) and chloroform. The LacZ assay is performed at 30° C. Samples are preheated, the assay initiated by addition of 200 μl ortho-nitro-phenyl-β-galactosidase (4 mg/ml) and stopped by addition of 500 μl of 1 M Na₂CO₃ when the sample had turned slightly yellow. The release of ortho-nitrophenol is subsequently determined as the change in optical density at 420 nm. The specific activities are reported in Miller Units (MU) [A420/(min*ml*A600)]. A regulatory element with an activity above 10,000 MU is considered strong and a regulatory element with an activity below 3,000 MU is considered weak, what is in between has intermediate strength. An example of a strong regulatory element is the PgIpF promoter with an activity of approximately 14.000 MU and an example of a weak promoter is Plac which when induced with IPTG has an activity of approximately 2300 MU.

Alternatively, if there is a need for balancing the expression level of one or more proteins to optimize the production it may be beneficial to use a promoter with the desired strength, e.g., middle or low strength. Table 0 below lists a series of wildtype and recombinant promoters according to their strength relative to the PgIpF promoter.

TABLE 0
Promoter sequences according to strength

| | % activity | | Seq ID |
|---|---|---|---|
| Promoter name | relative to PglpF* | Strength | in appl. |
| PmglB_70UTR_SD8 | 291% | high | 10 |
| PmglB_70UTR_SD10 | 233-281% | high | 11 |
| PmglB_54UTR | 197% | high | 12 |
| Plac_70UTR | 182-220% | high | 13 |
| PmglB_70UTR_SD9 | 180-226% | high | 14 |
| PmglB_70UTR_SD4 | 153%-353% | high | 15 |
| PmglB_70UTR_SD5 | 146-152% | high | 16 |
| PglpF_SD4 | 140-161% | high | 17 |
| PmglB_70UTR_SD7 | 127-173% | high | 18 |
| PmglB_70UTR | 124-234% | high | 19 |
| PglpA_70UTR | 102-179% | high | 20 |
| PglpT_70UTR | 102-240% | high | 21 |
| PgatY_70UTR | 112% | high | 22 |
| PglpF | 100% | high | 23 |
| PglpF_SD10 | 88-96% | high | 24 |
| PglpF_SD5 | 82-91% | high | 25 |
| PglpF_SD8 | 81-82% | high | 26 |
| PmglB_16UTR | 78-171% | high | 27 |
| PglpF_SD9 | 73-93% | middle | 28 |
| PglpF_SD7 | 47-57% | middle | 29 |
| PglpF_SD6 | 46-47% | middle | 30 |
| PglpA_16UTR | 38-64% | middle | 31 |
| Plac | 15-28% | low | 32 |
| PglpF_SD3 | 9% | low | 33 |
| PglpF_SD1 | 5% | low | 34 |

*The promoter activity is assessed in the LacZ assay described below with the PglpF promoter run as positive reference in the same assay. To compare across assays the activity is calculated relative to the PglpF promoter, a range indicates results from multiple assays.

The promoter may be of heterologous origin, native to the genetically modified cell or it may be a recombinant promoter, combining heterologous and/or native elements.

In one or more exemplary embodiment(s), the promoter sequence is selected from the group of promoters consisting of Plac, PgIpF, PgIpA, PgIpT or PmgIB and variants thereof.

In one or more exemplary embodiments, the promoter selected from the group consisting of PgIpF (SEQ ID NO: 23), PgIpT_70UTR (SEQ ID NO: 21), PgatY_70UTR (SEQ ID NO: 22), Plac_70UTR (SEQ ID NO: 13), PmgIB_54UTR (SEQ ID NO: 12), PmgIB_70UTR (SEQ ID NO: 19), PgIpA_70UTR (SEQ ID NO: 20), or variants thereof. Specifically, the variants disclosed in table 0 are preferred.

In a currently preferred embodiment, the promoter sequence is selected from the group consisting of strong promoters PmgIB_70UTR_SD8 (SEQ ID NO: 10), PmgIB_70UTR_SD10 (SEQ ID NO: 11), PmgIB_54UTR (SEQ ID NO: 12), Plac_70UTR (SEQ ID NO: 13), PmgIB_70UTR_SD9 (SEQ ID NO: 14), PmgIB_70UTR_SD4 (SEQ ID NO: 15), PmgIB_70UTR_SD5 (SEQ ID NO: 16), PgIpF_SD4 (SEQ ID NO: 17), PmgIB_70UTR_SD7 (SEQ ID NO: 18), PmgIB_70UTR (SEQ ID NO: 19), PgIpA_70UTR (SEQ ID NO: 20), PgIpT_70UTR (SEQ ID NO: 21), PgatY_70UTR (SEQ ID NO: 22), PgIpF (SEQ ID NO: 23), PgIpF_SD10 (SEQ ID NO: 24), PgIpF_SD5 (SEQ ID NO: 25), PgIpF_SD8 (SEQ ID NO: 26), PmgIB_16UTR (SEQ ID NO: 27).

In preferred embodiments, the overexpression of the nucleic acid sequences encoding enzymes of the UPD-GlcNAc pathway is under control of the PgIpF (SEQ ID NO: 23) promoter or another strong promoter selected from table 0.

In one embodiment the nucleic acid encoding GlmM is under control of the PgIpF (SEQ ID NO: 23) promoter or another strong promoter selected from table 0. Preferably, by substituting the native promoter with the stronger promoter.

In one embodiment the nucleic acid encoding GlmU is under control of the PgIpF (SEQ ID NO: 23) promoter or another strong promoter selected from table 0. Preferably, by substituting the native promoter with the stronger promoter.

In one embodiment the nucleic acid encoding GlmS is under control of the PgIpF (SEQ ID NO: 23) promoter or another strong promoter selected from table 0. Preferably, by substituting the native promoter with the stronger promoter.

The term “sequence identity” as used herein describes the relatedness between two amino acid sequences or between two nucleotide sequences, i.e., a candidate sequence (e.g., a sequence of the disclosure) and a reference sequence (such as a prior art sequence) based on their pairwise alignment. For purposes of the present disclosure, the sequence identity between two amino acid sequences is determined using the Needleman-Wunsch algorithm (Needleman and Wunsch, 1970, J. Mol. Biol. 48:443-453) as implemented in the Needle program of the EMBOSS package (EMBOSS: The European Molecular Biology Open Software Suite, Rice et al., 2000, Trends Genet. 16:276-277), preferably version 5.0.0 or later (available at https://www.ebi.ac.uk/Tools/psa/emboss needle/). The parameters used are gap open penalty of 10, gap extension penalty of 0.5, and the EBLOSUM62 (EMBOSS version of 30 BLOSUM62) substitution matrix. The output of Needle labelled “identity” (obtained using the-nobrief option) is used as the percent identity. Generally sequence identity may be calculated as follows: (Identical Residues×100)/(Length of Aligned region).

For purposes of the present disclosure, the sequence identity between two nucleotide sequences is determined using the Needleman-Wunsch algorithm (Needleman and Wunsch, 1 970, supra) as implemented in the Needle program of the EMBOSS package (EMBOSS: The European Molecular Biology Open Software Suite, Rice et al., 2000, Trends Genet. 16:276-277), 10 preferably version 5.0.0 or later. The parameters used are gap open penalty of 10, gap extension penalty of 0.5, and the DNAFULL (EMBOSS version of NCBI NUC4.4) substitution matrix. The output of Needle labelled “identity” (obtained using the-nobrief option) is used as the percent identity. Generally sequence identity may be calculated as follows: (Identical Deoxyribonucleotides×100)/(Length of Aligned region).

Cells Capable of Producing N-Acetylated- and/or Sialylated Molecules

In embodiments, the cell according to the disclosure is capable of producing one or more oligosaccharides, glycoproteins or glycolipids, in particular one or more N-acetylated- and/or sialylated oligosaccharides or one or more N-acetylated- and/or sialylated glycoproteins or one or more N-acetylated- and/or sialylated glycolipids.

The expression “N-acetylated oligosaccharide(s)” or “N-acetylated protein(s)” or “N-acetylated lipid(s)” relates to oligosaccharide(s), protein(s) or lipid(s) containing at least one N-acetylglucosamine moiety, also described as a N-acetylglucosamine containing molecule (e.g., oligosaccharide, lipid or protein). Suitable examples of N-acetylated oligosaccharides are lacto-N-triose II (LNT-II), lacto-N-neotetraose (LNnT), lacto-N-tetraose (LNT), para-lacto-N-neohexaose (para-LNnH), disialyllacto-N-tetraose (DSLNT) or any combination thereof. Other examples are lacto-N-hexaose, lacto-N-neohexaose, para-lacto-N-hexaose, para-lacto-N-neohexaose, lacto-N-octaose, lacto-N-neooctaose, iso-lacto-N-octaose, para-lacto-N-octaose and lacto-N-decaose or other HMOs listed below containing an LNT-II backbone. Other examples of N-acetylated oligosaccharides are polysaccharides such as chitin, chitosan hyaluronic acid, chondroitin sulfate or heparan sulfate.

The expression “sialylated oligosaccharide(s)” or “sialylated protein(s)” or “sialylated lipid(s)” relates to oligosaccharide(s), protein(s) or lipid(s) containing at least one N-Acetylneuraminic acid (Neu5Ac) or at least one O-Acetylneuraminic acid (Neu9Ac) moiety. Suitable examples of N-acetylated oligosaccharides include 3′-sialyllactose (3′SL), 6′-sialyllactose (6′SL) and 3-fucosyl-3′-sialyllactose (FSL), sialyl-Lewis-X, 6′-sialyllacto-N-tetraose b (LST-b), 6′-sialyllacto-N-neotetraose (LST-c), 3′-sialyllacto-N-neotetraose (LST-d) or other HMOs listed below containing a sialyl moiety.

Examples of N-acetylated and/or sialylated glycoproteins are for example mucins. Glyco-conjugated proteins are also increasingly explored for their therapeutic potential such as monoclonal antibodies engineered to contain specific glycosylation patterns, including GlcNAc residues, which can be crucial for the targeting and efficacy of the monoclonal antibodies.

Examples of N-acetylated and/or sialylated glycolipids are for example sialylated liposomes or gangliosides or glycosphingolipids which both are decorated with sialic acid and N-acetylglucosamine in varying amounts.

In embodiments the N-acetylated- and/or sialylated molecule is selected from the group of oligosaccharides, glycoproteins and glycolipids. If the molecule is N-acetylated then, preferably, at least one N-acetylation in the molecule is a N-acetylglucosamine moiety.

In embodiments the cell according to the disclosure is capable of producing one or more oligosaccharides is selected from the group consisting of: LNT-II, LNT, LNnT, LNH, LNnH, pLNH, pLNnH, LNFP-I, LNFP-II, LNFP-III, LNFP-V, LNFP-V, LNFP-VI, LNDFH-I, LNDFH-II, LNDFH-III, F-pLNnH, DF-LNH a, DF-LNH b, DF-LNH c, DF-pLNnH, TF-pLNnH, FDS-LNH, TF-LNH, DS-LNH, F-LNH-I, F-LNH-II, F-LNH-III, FLST-a (S-LNFP-II), FLST-b, pLNnH, FLST-c, FLST-d, FSL, sialyl-Lewis-X, 3′SL, 6′SL, LST-a, LST-b, LST-c, LST-d, DSLNT, SLNH, SLNH-II, and any mixture thereof or polysaccharides selected from the group consisting of chitin, chitosan, hyaluronic acid, chondroitin sulfate or heparan sulfate.

In a preferred embodiment, the cell according to the disclosure is capable of producing one or more human milk oligosaccharides (HMOs), in particular one or more N-acetylated- and/or sialylated HMOs.

The term “human milk oligosaccharide” or “HMO” in the present context means a complex carbohydrate found in human breast milk. The HMOs have a core structure comprising a lactose unit at the reducing end that can be elongated by one or more beta-N-acetyl-lactosaminyl and/or one or more beta-lacto-N-biosyl unit, and this core structure can be substituted by an alpha-L-fucopyranosyl and/or an alpha-N-acetyl-neuraminyl (sialyl) moiety. HMO structures are e.g., disclosed by Xi Chen in Chapter 4 of Advances in Carbohydrate Chemistry and Biochemistry, 2015, Vol. 72.

HMOs can be non-acidic (or neutral) or acidic. Neutral HMOs are devoid of a sialyl residue and acidic HMOs have at least one sialyl residue in their structure.

The non-acidic (or neutral) HMOs can be fucosylated or non-fucosylated.

In embodiments, the HMO produced by the genetically modified cell is an HMO comprising a N-acetylglucosamine (GlcNAc) moiety or a sialic acid (Neu5Ac) moiety. The production of oligosaccharides with these moieties is dependent on a functional UDP-GlcNAc pathway.

Examples of GlcNAc containing neutral non-fucosylated HMOs include lacto-N-triose II (LNT-II) lacto-N-tetraose (LNT), lacto-N-neotetraose (LNnT), lacto-N-neohexaose (LNnH), para-lacto-N-neohexaose (pLNnH), para-lacto-N-hexaose (pLNH) and lacto-N-hexaose (LNH).

Examples of GlcNAc containing neutral fucosylated HMOs include lacto-N-fucopentaose I (LNFP-I), lacto-N-difucohexaose I (LNDFH-I), lacto-N-fucopentaose II (LNFP-II), lacto-N-fucopentaose III (LNFP-III), lacto-N-difucohexaose III (LNDFH-III), fucosyl-lacto-N-hexaose II (FLNH-II), lacto-N-fucopentaose V (LNFP-V), lacto-N-difucohexaose II (LNDFH-II), fucosyl-lacto-N-hexaose I (FLNH-I), fucosyl-para-lacto-N-hexaose I (FpLNH-I), fucosyl-para-lacto-N-neohexaose II (F-pLNnH II) and fucosyl-lacto-N-neohexaose (FLNnH).

Examples of acidic HMOs comprising a sialic acid moiety include 3′-sialyllactose (3′SL), 6′-sialyllactose (6′SL) and 3-fucosyl-3′-sialyllactose (FSL).

Examples of acidic HMOs comprising a sialic acid moiety and a GlcNAc moiety include 3′-O-sialyllacto-N-tetraose a (LST-a), fucosyl-LST a (FLST-a), 6′-O-sialyllacto-N-tetraose b (LST-b), fucosyl-LST b (FLST-b), 6′-O-sialyllacto-N-neotetraose (LST-c), fucosyl-LST c (FLST-c), 3′-O-sialyllacto-N-neotetraose (LST-d), fucosyl-LST d (FLST-d), sialyl-lacto-N-hexaose (SLNH), sialyl-lacto-N-neohexaose I (SLNH-I), sialyl-lacto-N-neohexaose II (SLNH-II) and disialyl-lacto-N-tetraose (DSLNT).

In a particular embodiment, the cell is capable of producing one or more human milk oligosaccharides (HMOs) selected from the group consisting of: LNT, LNT-II, LNnT, LNH, LNnH, pLNH, pLNnH, LNFP-I, LNFP-II, LNFP-III, LNFP-V, LNFP-VI, LNDFH-I, LNDFH-II, LNDFH-III, F-pLNnH, DF-LNH a, DF-LNH b, DF-LNH c, DF-pLNnH, TF-pLNnH, FDS-LNH, TF-LNH, DS-LNH, F-LNH-I, F-LNH-II, F-LNH-III, FLST-a (S-LNFP-II), FLST-b, pLNnH, FLST-c, FLST-d, FSL, 3′SL, 6′SL, LST-a, LST-b, LST-c, LST-d, DSLNT, SLNH, SLNH-II, and any mixture thereof.

A genetically modified cell according to the present disclosure that is capable of producing one or more human milk oligosaccharides (HMOs) comprises at least one recombinant nucleic acid sequence encoding a glycosyltransferase activity capable of transferring a glycosyl moiety from an activated sugar to an acceptor oligosaccharide.

In the context of the present disclosure, an acceptor oligosaccharide is an oligosaccharide that can act as a substrate for a glycosyltransferase capable of transferring a glycosyl moiety from a glycosyl-donor to the acceptor oligosaccharide. The glycosyl-donor can be a nucleotide-activated sugar such as UDP-N-acetylglucosamine (UDP-GlcNAc), potentially in combination with further glycosyl doners as described below. The acceptor oligosaccharide can be a precursor for making a more complex HMO (composed of 4 monosaccharides or more). The acceptor oligosaccharide can therefore also be termed precursor molecule.

In the present context, the acceptor oligosaccharide for the glycosyltransferase can be lactose, lacto-N-triose II (LNT-II), lacto-N-tetraose (LNT), lacto-N-neotetraose (LNnT), 2′-fucosyllactose (2′FL), 3-fucosyllactose (3FL), 3′-sialyllactose (3′SL) or 6′-salyllactose (6′SL). In a preferred embodiment the acceptor molecule is lactose.

### Glycosyltransferases

As indicated above, the genetically modified cell according to the present disclosure is capable of producing one or more oligosaccharides, such as human milk oligosaccharides (HMOs) with a GlcNAc or sialic acid moiety and comprises at least one recombinant nucleic acid sequence encoding at least one glycosyltransferase capable of transferring a glycosyl residue from a glycosyl-donor to an acceptor oligosaccharide to synthesize a human milk oligosaccharide. The nucleic acid sequence encoding the one or more expressed glycosyltransferase(s) may be integrated into the genome (by chromosomal integration) of the genetically engineered cell, or alternatively, it may be comprised in a plasmid and expressed as plasmid-borne.

The genetically modified cell according to the present disclosure may comprise at least two recombinant nucleic acid sequences encoding two different glycosyltransferases capable of transferring a glycosyl residue from a glycosyl-donor to an acceptor oligosaccharide.

The one or more glycosyltransferase is preferably selected from the group of enzymes having the activity of an α-1,2-fucosyltransferase, α-1,3-fucosyltransferase, α-1,3/4-fucosyltransferase, α-1,4-fucosyltransferase α-2,3-sialyltransferase, α-2,6-sialyltransferase, β-1,3-N-acetylglucosaminyltransferase, β-1,6-N-acetylglucosaminyltransferase, β-1,3-galactosyltransferase and β-1,4-galactosyltransferase, described in more detail below.

For production of chitooligosaccharides, such as chitin and chitosan, the glycosyltransferase may be chitin synthase (NodC) (see for example CN114990174) and for the production of hyaluronic acid the hyaluronan synthase which also possess glycosyltransferase activity may be used (see for example Widner et al 2005 Appl Environ Microbiol. 71 (7): 3747-3752 and Sze et al 2016, 3 Biotech. 6 (1): 67).

### β-1,3-N-Acetyl-Glucosaminyltransferase

A β-1,3-N-acetyl-glucosaminyltransferase is any protein which comprises the ability of transferring the N-acetyl-glucosamine of UDP-N-acetyl-glucosamine to lactose or another acceptor molecule, in a beta-1,3-linkage. Preferably, a β-1,3-N-acetyl-glucosaminyltransferase used herein does not originate in the species of the genetically engineered cell i.e., the gene encoding the β-1,3-galactosyltransferase is of heterologous origin. Non-limiting examples of β-1,3-N-acetyl-glucosaminyltransferase are given in Table 1. Variants of β-1,3-N-acetyl-glucosaminyltransferases may also be useful, preferably such variants that are at least 80%, such as at least 85%, such as at least 90%, such as at least 95% identical to one of the β-1,3-N-acetyl-glucosaminyltransferase given in Table 1.

TABLE 1
List of β-1,3-N-acetyl-glucosaminyltransferases

| Protein | | | |
|---|---|---|---|
| Name | GenBank ID | origin | Exemplary HMO products |
| lgtA | WP_033911473.1 | Neisseria | LNT-II, LNT, LNnT, LNFP-I, LNFP-II, |
| | WP_002248149 | meningitidis | LNFP-III, LNFP-V, LNFP-VI, LNDFH-I, |
| | AAF42258.1 | | LNDFH-II, LNDFH-III, pLNH, F-pLNH I, |
| | ELK60643.1 | | pLNnH, LST a, LST b, LST c, DSLNT |
| lgtA | AAK70338.1 | Neisseria | LNT-II, LNT, LNnT, LNFP-I, LNFP-II, |
| | ACF31229.1 | gonorrhoeae | LNFP-III, LNFP-V, LNFP-VI, LNDFH-I, |
| | | | LNDFH-II, LNDFH-III, pLNH, F-pLNH I, |
| | | | pLNnH |
| lgtA | AAK02595.1 | Pasteurella | LNT-II, LNT, LNnT, LNFP-I, LNFP-II, |
| | | multocida | LNFP-III, LNFP-V, LNFP-VI, LNDFH-I, |
| | | | LNDFH-II, LNDFH-III, pLNH, F-pLNH I, |
| | | | pLNnH |
| lgtA | EEZ72046.1 | Neisseria | LNT-II, LNT, LNnT, LNFP-I, LNFP-II, |
| | | cinerea | LNFP-III, LNFP-V, LNFP-VI, LNDFH-I, |
| | | | LNDFH-II, LNDFH-III, pLNH, F-pLNH I, |
| | | | pLNnH |
| HD0466 | WP_010944479.1 | Haemophilus | LNT-II, LNT, LNnT, LNFP-I, LNFP-II, |
| | | ducreyi | LNFP-III, LNFP-V, LNFP-VI, LNDFH-I, |
| | | | LNDFH-II, LNDFH-III, pLNH, F-pLNH I, |
| | | | pLNnH, LST a, LST b, LST c, DSLNT |
| PmnagT | WP_014390683.1 | Pasteurella | LNT-II, LNT, LNnT, LNFP-I, LNFP-II, |
| | | multocida | LNFP-III, LNFP-V, LNFP-VI, LNDFH-I, |
| | | | LNDFH-II, LNDFH-III, pLNH, F-pLNH I, |
| | | | pLNnH, LST a, LST b, LST c, DSLNT |

In embodiments, the genetically modified cell is capable of producing an HMO and comprises i) a deleted or dysfunctional yhbJ gene and ii) a β-1,3-N-acetyl-glucosaminyltransferase.

### Heterologous β-1,6-N-Acetylglucosaminyltransferase

A heterologous β-1,6-N-acetyl-glucosaminyl-transferase is any protein which comprises the ability of transferring the N-acetyl-glucosamine of UDP-N-acetyl-glucosamine to an acceptor molecule, in a β-1,6-linkage. A β-1,6-N-acetyl-glucosaminyl-transferase used herein does not originate in the species of the genetically engineered cell, i.e. the gene encoding the β-1,6-galactosyltransferase is of heterologous origin. An example of a β-1,6-N-acetyl-glucosaminyl-transferase is Csp2 from Chryseobacterium sp. KBW03 (NCBI accession Nr. WP_22844786.1) or a variant thereof which for example can produce LNH or LNnH.

### β-1,3-Galactosyltransferase

A β-1,3-Galactosyltransferase is any protein that comprises the ability of transferring the galactose of UDP-Galactose to a N-acetyl-glucosaminyl moiety to an acceptor molecule in a β-1,3-linkage. Preferably, a β-1,3-galactosyltransferase used herein does not originate in the species of the genetically engineered cell, i.e., the gene encoding the β-1,3-galactosyltransferase is of heterologous origin. Non-limiting examples of β-1,3-galactosyltransferases are given in Table 2. Variants of β-1,3-galactosyltransferases may also be useful, preferably such variants that are at least 80%, such as at least 85%, such as at least 90%, such as at least 95% identical to one of the β-1,3-galactosyltransferases given in Table 2.

TABLE 2
List of β-1,3-glycosyltransferases

| Protein Name | GenBank ID | Origin |
|---|---|---|
| WbgO | WP_000582563.1 | E. coli 055: H7 |
| Jhp0563 | AEZ55696.1 | Helicobacter pylori |
| GalTK | WP_111735921.1 | Helicobacter pylori ATCC 43504 |
| CvB3GalT | WP_080969100.1 | Chromobacterium violaceum |

In embodiments, the genetically modified cell is capable of producing an HMO and comprises i) a deleted or dysfunctional yhbJ gene, and ii) a β-1,3-N-acetyl-glucosaminyltransferase and iii) a β-1,3-galactosyltransferases.

In further embodiments, the HMO produced by said cell is selected from LNT, LNFP-I, LNFP-II, LNFP-V, LNDFH-I, LNDFH-II, LST-a, FLST-a, LNH, pLNH, pLNH2, FLNH-I, FLNH-II, F-LNH-III.

### β-1,4-Galactosyltransferase

A β-1,4-Galactosyltransferase is any protein that comprises the ability of transferring the galactose of UDP-Galactose to a N-acetyl-glucosaminyl moiety. Preferably, a β-1,4-galactosyltransferase used herein does not originate in the species of the genetically engineered cell, i.e., the gene encoding the β-1,4-galactosyltransferase is of heterologous origin. Non-limiting examples of β-1,4-galactosyltransferases are given in Table 3. β-1,4-galactosyltransferases variants may also be useful, preferably such variants are at least 80%, such as at least 85%, such as at least 90%, such as at least 95% identical to one of the β-1,4-galactosyltransferases in Table 3.

TABLE 3
List of beta-1,4-glycosyltransferases

| Protein Name | GenBank ID | Origin |
|---|---|---|
| GalT | WP_001262061.1 | Helicobacter pylori |
| LgtB | AAF42257.1 | Neisseria meningitidis MC58 |
| Aph | WP_005701792.1 | Aggregatibacter aphrophilus |

In embodiments, the genetically modified cell is capable of producing an HMO and comprises i) a deleted or dysfunctional yhbJ gene, and ii) a β-1,3-N-acetyl-glucosaminyltransferase and iii) a β-1,4-galactosyltransferases.

In further embodiments the HMO produced by said cell is selected from LNnT, LNFP-III, LNFP-VI, LNDFH-III, DF-LNH a, DF-LNH b, DF-LNH c, DF-pLNnH, TF-pLNnH, FDS-LNH, TF-LNH, DS-LNH, F-LNH-I, F-LNH-II, F-LNH-III, LST-c, FLST-c, LNnH or pLNnH.

### Alpha-1,2-Fucosyltransferase

An α-1,2-fucosyltransferase is a protein that comprises the ability to catalyze the transfer of fucose from a donor substrate, for example, GDP-fucose, to an acceptor molecule in an alpha-1,2-linkage. Preferably, an alpha-1,2-fucosyltransferase used herein does not originate in the species of the genetically engineered cell, i.e., the gene encoding the alpha-1,2-fucosyltransferase is of heterologous origin. Non-limiting examples of alpha-1,2-fucosyltransferase are given in Table 4. Variants of alpha-1,2-fucosyltransferases may also be useful, preferably such variants are at least 80%, such as at least 85%, such as at least 90%, such as at least 95% identical to one of the alpha-1,2-fucosyltransferase in Table 4.

TABLE 4
List of α-1,2-fucosyltransferases

| Protein Name | GenBank ID | Origin |
|---|---|---|
| FutC | WP_080473865.1 | Helicobacter pylori |
| | | ATCC 26695 |
| wbgL | ADN43847 | E. coli O126 |
| Smob | WP_126455392.1 | Sulfuriflexus mobilis |
| FucT2_HpUA802 | AAC99764.1 | Helicobacter pylori |
| FucT2_EcO126t | ABE98421.1 | Escherichia coli |
| FucT2_Hm12198 | CBG40460.1 | Helicobacter mustelae 12198 |
| FucT2_Pm9515 | ABM71599.1 | Brochlorococcus marinus str. |
| | | MIT 9515 |
| FucT2_HpF57 | BAJ59215.1 | Helicobacter pylori F57 |
| FucT54 | ADE13114.1 | Sideroxydans lithotrophicus |
| | | ES-11 |
| Mtun | WP_031437198.1 | Methylobacter tundripaludum |

### Alpha-1,3-Fucosyltranferase

An alpha-1,3-fucosyltranferase refer to a glycosyltransferase that catalyzes the transfer of fucose from a donor substrate for example, GDP-fucose, to an acceptor molecule in an alpha-1,3-linkage. Preferably, an alpha-1,3-fucosyltransferase used herein does not originate in the species of the genetically engineered cell, i.e., the gene encoding the alpha-1,3-fucosyltransferase is of heterologous origin. Non-limiting examples of alpha-1,3-fucosyltransferase are given in Table 5. Variants of alpha-1,3-fucosyltransferase may also be useful, preferably such variants are at least 80%, such as at least 85%, such as at least 90%, such as at least 95% identical to one of the alpha-1,3-fucosyltransferase in Table 5.

TABLE 5
List of α-1,3-fucosyltransferases

| Protein Name | GenBank ID | Origin |
|---|---|---|
| FucT | AAB81031.1 | Helicobacter pylori |
| | | NCTC 11639 |
| FutA | WP_000487428.1 | Helicobacter pylori |
| | | ATCC 26695 |
| CafC | WP_007483358.1 | Bacteroides nordii |
| FucT6_3_Bf | CAH09151.1 | Bacteroides fragilis |
| | | NCTC 9343 |
| FucT7_3_Bf | CAH09495.1 | Bacteroides fragilis |
| | | NCTC 9343 |
| FucT_3_Am | ACD04596.1 | Akkermansia muciniphila |
| | | ATCC BAA-835 |
| MAMA_R764 | AGC02224.1 | Acanthamoeba polyphaga |
| | | moumouvirus |
| Mg791 | AEQ33441.1 | Megavirus chiliensis |
| Moumou_00703 | YP_007354660.1 | Acanthamoeba polyphaga |
| | | moumouvirus |

### Alpha-1,3/4-Fucosyltransferase

An alpha-1,3/4-fucosyltransferase refers to a glycosyltransferase that catalyzes the transfer of fucose from a donor substrate for example, GDP-fucose, to an acceptor molecule in an alpha-1,3- or alpha-1,4-linkage. Preferably, an alpha-1,3/4-fucosyltransferase used herein does not originate in the species of the genetically engineered cell, i.e. the gene encoding the alpha-1,3/4-fucosyltransferase is of heterologous origin. Non-limiting examples of alpha-1,3/4-fucosyltransferase are given in Table 6. Variants of alpha-1,3/4-fucosyltransferases may also be useful, preferably such variants are at least 80%, such as at least 85%, such as at least 90%, such as at least 95% identical to one of the alpha-1,3/4-fucosyltransferase in Table 6.

TABLE 6
List of α-1,3/4-fucosyltransferase

| Protein | | |
|---|---|---|
| Name | GenBank ID | Origin |
| FucTIII | AY450598.1 | Helicobacter pylori strain |
| | | DSM 6709 |
| FutA | WP_000487428.1 | Helicobacter pylori |
| | | ATCC 26695 |
| Med1 | WP_087337236.1 | Mediterranea sp. An20 |

### Alpha-2,3-Sialyltransferase

An alpha-2,3-sialyltransferase refers to a glycosyltransferase that catalyzes the transfer of sialyl from a donor substrate for example, CMP-N-acetylneuraminic acid, to an acceptor molecule in an alpha-2,3-linkage. Preferably, an alpha-2,3-sialyltransferase used herein does not originate in the species of the genetically engineered cell i.e., the gene encoding the alpha-2,3-sialyltransferase is of heterologous origin. Non-limiting examples alpha-2,3-sialyltransferase are given in Table 7. Variants of alpha-2,3-sialyltransferases may also be useful, preferably such variants are at least 80%, such as at least 85%, such as at least 90%, such as at least 95% identical to one of the alpha-2,3-sialyltransferase in Table 7.

TABLE 7
List of alpha-2,3-sialyltransferases

| Protein Name | GenBank ID | Origin |
|---|---|---|
| Avi | WP_115249238.1 | Avibacterium avium |
| Ccol | WP_075498955.1 | Campylobacter coli |
| Ccol2 | EAH6554614.1 | Campylobacter coli |
| Chepa | WP_066776435.1 | Campylobacter hepaticus |
| Cinf1 | WP_011272254.1 | Haemophilus influenzae |
| Cjej1 | EBD1936710.1 | Campylobacter jejuni |
| Clari1 | EGK8106227.1 | Campylobacter lari |
| CstI | AAF13495.1 | Campylobacter jejuni |
| CstII | AAF31771.1 | Campylobacter jejuni |
| Csub1 | WP_039664428.1 | Campylobacter subantarcticus |
| MhnNBse | WP_176810284.1 | Mannheimia (multispecies) |
| Neigon | AAW89748.1 | Neisseria gonorrhoeae FA 1090 |
| Nst | AAC44541.1 | Neisseria meningitidis MC58 |
| PM70 | AAK03258.1 | Pasteurella multocida subsp. |
| | | multocida str. Pm70 |
| PmN | WP_005726268.1 | Pasteurella (multispecies) |
| Pmult | WP_005753497.1 | Pasteurela multocida |
| Poral | WP_101774487.1 | Pasteurella oralis |
| Poral_2 | WP_101774701.1 | Pasteurella oralis |

In embodiments, the genetically modified cell is capable of producing an HMO and comprises i) a deleted or dysfunctional yhbJ gene, and ii) an alpha-2,3-sialyltransferase.

In further embodiments, the HMO produced by said cell is selected from 3′SL, FSL, LST-a, DS-LNT and FLST-a.

### Alpha-2,6-Sialyltransferase

An alpha-2,6-sialyltransferase refers to a glycosyltransferase that catalyzes the transfer of sialyl from a donor substrate for example, CMP-N-acetylneuraminic acid, to an acceptor molecule in an alpha-2,6-linkage. Preferably, an alpha-2,6-sialyltransferase used herein does not originate in the species of the genetically engineered cell, i.e., the gene encoding the alpha-2,6-sialyltransferase is of heterologous origin. Non-limiting examples alpha-2,6-sialyltransferases are given in Table 8. Variants of alpha-2,6-sialyltransferases may also be useful, preferably such variants are at least 80%, such as at least 85%, such as at least 90%, such as at least 95% identical to one of the alpha-2,6-sialyltransferase in Table 8.

TABLE 8
List of alpha-2,6-sialyltransferases

| Protein | | |
|---|---|---|
| Name | GenBank ID | Origin |
| HAC1268 | STP03859.1 | Helicobacter acinonychis str. Sheeba |
| Shal | WP_108944675.1 | Shewanella halifaxensis |
| Pts6-119 | AB500947.1 | Photobacterium leiognathi |
| | | JT-SHIZ-119 |
| Pts6-224 | BAF92026.1 | Photobacterium sp JT-ISH-224 |
| PiST6_145 | BAF91416.1 | Photobacterium leiognathi |

In embodiments, the genetically modified cell is capable of producing an HMO and comprises i) a deleted or dysfunctional yhbJ gene, and ii) an α-2,6-sialyltransferase.

In further embodiments, the HMO produced by said cell is selected from 6′SL, LST-c or FLST-C.

### Preferred Embodiments of Cells Capable of Producing Human Milk Oligosaccharides (HMO)

In a preferred embodiment, the genetically modified cell is capable of producing an HMO mixture comprising LNT-II, LNnT, and pLNnH, and comprises the following modifications:
a) deleted or dysfunctional yhbJ gene;
b) a recombinant beta-1,3-N-acetyl-glucosaminyltransferase;
c) a recombinant beta-1,4-galactosyltransferase; and
d) optionally overexpression of phosphoglucosamine mutase (GlmM);
e) optionally overexpression of N-acetylglucosamine-1-phosphate uridyltransferase (GlmU), and/or overexpression of glucosamine-6-phopshate synthase (GlmS).

In the aforementioned embodiment, preferably, at least 15%, such as at least 20%, such as at least 25% of the molar content of the total HMO produced by the cell is pLNnH.

In a preferred embodiment, the genetically modified cell is capable of producing an HMO mixture comprising LNT-II, LNT, and pLNH2, and comprises the following modifications:
a) deleted or dysfunctional yhbJ gene;
b) overexpression of phosphoglucosamine mutase (GlmM);
c) a recombinant beta-1,3-N-acetyl-glucosaminyltransferase;
d) a recombinant beta-1,4-galactosyltransferase; and
e) optionally overexpression of N-acetylglucosamine-1-phosphate uridyltransferase (GlmU), and/or overexpression of glucosamine-6-phopshate synthase (GlmS).

In the aforementioned embodiment, preferably the LNT formation is increased by at least 50% compared to a cell that does not contain the modifications of a), b) and e).

In a preferred embodiment, the genetically modified cell is capable of producing a sialylated oligosaccharide, such as a human milk oligosaccharide, and comprises the following modifications:
a) deleted or dysfunctional yhbJ gene;
b) an alpha-2,3-sialyltransferase or alpha-2,6-sialyltransferase and
c) a biosynthetic pathway for making a sialic acid sugar nucleotide, and
d) optionally overexpression of N-acetylglucosamine-1-phosphate uridyltransferase (GlmU).

In the aforementioned embodiment, the genetically modified cell produces a sialylated human milk oligosaccharide selected from the group consisting of 3′SL, 6′SL, LST-a, LST-b, LST-c and DS-LST, preferably the sialylated HMO is 3′SL or 6′SL. Even more preferably the modified cell comprises an alpha-2,3-sialyltransferase and produces at least 1.5, such as 1.75, such as 2-fold more 3′SL compared to a cell that does not contain the modifications of a) and d).

In a preferred embodiment, the genetically modified cell is capable of producing LNT-II and comprises the following modifications:
a) deleted or dysfunctional yhbJ gene;
b) overexpress phosphoglucosamine mutase (GlmM);
c) a recombinant beta-1,3-N-acetyl-glucosaminyltransferase; and
d) optionally overexpression of N-acetylglucosamine-1-phosphate uridyltransferase (GlmU), and/or overexpression of glucosamine-6-phopshate synthase (GlmS).

In the aforementioned embodiment, preferably the LNT-II formation is increased by at least 75%, such as 100% compared to a cell that does not contain the modifications of a), b) and e).

### Further Glycosyl-Donors and Nucleotide Activated Sugars

Apart from the capability of producing UDP-N-acetylglucosamine (UDP-GlcNAc), the cell according to the disclosure may be capable of producing further glycosyl-donor compounds to produce, for example, fucosylated or sialylated HMOs. Hence, the modified cell according to the disclosure may have the genetic capabilities to produce further glycosyl-donor compounds such as nucleotide-activated sugars.

The further glycosyl-donor compounds may be: UDP-galactose, GDP-fucose, CMP-N-acetylneuraminic acid (CMP-Neu5Ac), or any combination thereof. In Table 9 below, the HMOs are given that can be produced with said glycosyl-donors:

TABLE 9
Glycosyl-donor HMO product list

| Glycosyl-donor | Involved in the production of the following HMO products |
|---|---|
| UDP-GlcNAc | LNT, LNnT, LNFP-I, LNFP-II, LNFP-III, LNFP-V, LNFP-VI, LNDFH-I, |
| | LNDFH-II, LNDFH-III, LNH, LNnH, pLNH, pLNnH, F-pLNH-I, F-pLNH-II, |
| | F-pLNH-I, F-pLNnH-II, FLSTa, FLSTb, FLSTc, FLSTd, LSTa, LSTb, |
| | LSTc, LSTd, DSLNT, SLNH, SLNH-II, DF-LNH a, DF-LNH b, DF-LNH c, |
| | DF-pLNnH, TF-pLNnH, pLNnH, FDS-LNH, TF-LNH, DS-LNH, F-LNH-I, |
| | F-LNH-II, F-LNH-III |
| UDP-Gal | LNT, LNnT, LNFP-I, LNFP-II, LNFP-III, LNFP-V, LNFP-VI, LNDFH-I, |
| | LNDFH-II, LNDFH-III, LNH, LNnH, pLNH, pLNnH, F-pLNH-I, F-pLNH-II, |
| | F-pLNH-I, F-pLNnH-II, FLSTa, FLSTb, FLSTc, FLSTd, LSTa, LSTb, |
| | LSTc, LSTd, DSLNT, SLNH-I, SLNH-II, DF-LNH a, DF-LNH b, DF-LNH c, |
| | DF-pLNnH, TF-pLNnH, pLNnH, FDS-LNH, TF-LNH, DS-LNH, F-LNH-I, |
| | F-LNH-II, F-LNH-III |
| GDP-fucose | 2′FL, 3FL, DFL, LNFP-I, LNFP-II, LNFP-III, LNFP-V, LNFP-VI, LNDFH-I, |
| | LNDFH-II, LNDFH-III, F-LNH, F-LNnH, F-pLNH-I, F-pLNH-II, F-pLNH-I, |
| | F-pLNnH-II, FSL, FLSTa, FLSTb, FLSTc, FLSTd, DF-LNH a, DF-LNH b, |
| | DF-LNH c, DF-pLNnH, TF-pLNnH, pLNnH, FDS-LNH, TF-LNH, DS-LNH, |
| | F-LNH-I, F-LNH-II, F-LNH-III |
| CMP-Neu5Ac | 3′SL, 6′SL, FSL, FLSTa (S-LNFP-II), FLSTb, FLSTc, FLSTd, LSTa, LSTb, |
| | LSTc, LSTd, DSLNT, SLNH-I, SLNH-II, FDS-LNH, DS-LNH |

In one embodiment, the genetically modified cell may be capable of producing the further activated sugar nucleotides by a de novo pathway. In this regard, the further activated sugar nucleotide may be made by the cell under the action of enzymes involved in the de novo biosynthetic pathway in a stepwise reaction sequence starting from a simple carbon source like glycerol, sucrose, fructose or glucose.

The enzymes involved in the de novo biosynthetic pathway can be naturally present in the cell or introduced into the cell by means of gene technology or recombinant DNA techniques, all of them are parts of the general knowledge of the skilled person.

### Sialic Acid Sugar Nucleotide Synthesis Pathway

For the generation of sialylated molecules, such as sialylated HMOs the genetically modified cell comprises a sialic acid sugar nucleotide synthesis capability, i.e., the genetically modified cell comprises a biosynthetic pathway for making a sialate sugar nucleotide, such as CMP-N-acetylneuraminic acid (CMP-Neu5Ac) as glycosyl-donor for the sialyltransferases. E.g., the genetically modified cell comprises a sialic acid synthetic capability through provision of an exogenous UDP-GlcNAc 2-epimerase (e.g., neuC of Campylobacter jejuni (GenBank AAK91727.1) or equivalent (e.g., (GenBank CAR04561.1), a Neu5Ac synthase (e.g., neuB of C. jejuni (GenBank AAK91726.1) or equivalent, (e.g., Flavobacterium limnosediminis sialic acid synthase, GenBank WP_023580510.1), and/or a CMP-Neu5Ac synthetase (e.g., neuA of C. jejuni (GenBank AAK91728.1) or equivalent, (e.g., Vibrio brasiliensis CMP-sialic acid synthase, GenBank WP_006881452.1).

The UDP-GlcNAc is the substrate for the UDP-GlcNAc 2-epimerase (neuC), and for this reason the increased ability to produce UDP-GlcNAc as described herein is also expected to increase the formation of CMP-Neu5Ac.

In one or more examples, UDP-GlcNAc 2-epimerase, CMP-Neu5Ac synthetase, Neu5Ac synthase from Campylobacter jejuni, also referred to as neuBCA from Campylobacter jejuni or simply the neuBCA operon, may be plasmid borne or integrated into the genome of the genetically modified cell. Preferably, the sialic acid sugar nucleotide pathway is encoded by the nucleic acid sequence encoding neuBCA from Campylobacter jejuni (SEQ ID NO: 35) or a functional variant thereof having an amino acid sequence which is at least 80% identical, such as at least 85%, such as at least 90% or such as at least 99% to SEQ ID NO: 35.

Furthermore, the genetically modified cell may be deficient in the sialic acid catabolic pathway. By “sialic acid catabolic pathway” is meant a sequence of reactions, usually controlled, and catalysed by enzymes, which results in the degradation of sialic acid. An exemplary sialic acid catabolic pathway described hereafter is the E. coli pathway. In this pathway, sialic acid (Neu5Ac; N-acetylneuraminic acid) is degraded by the enzymes NanA (N-acetylneuraminic acid lyase) and NanK (N-acetylmannosamine kinase) and NanE (N-acetylmannosamine-6-phosphate epimerase), all encoded from the nanATEK-yhcH operon, and repressed by NanR (http://ecocyc.org/ECOLI).

### Colanic Acid Gene Cluster

For the production of fucosylated HMOs the colanic acid gene cluster is important to ensure presence of sufficient GDP-fucose. In Escherichia coli, GDP-fucose is an intermediate in the production of the extracellular polysaccharide colanic acid, a major oligosaccharide of the bacterial cell wall. In the context of the present disclosure, the colanic acid gene cluster encodes most of the enzymes involved in the de novo synthesis of GDP-fucose (gmd, wcaG, wcaH, wcaI, manB, manC), whereas one or several of the genes downstream of GDP-L-fucose, such as wcaJ, can be deleted to prevent conversion of GDP-fucose to colanic acid.

The de novo GDP-fucose pathway genes responsible for the formation of GDP-fucose comprises or consists of the following genes:
i) manA which encodes the protein mannose-6 phosphate isomerase (EC 5.3.1.8, UniProt accession nr. P00946), which facilitates the interconversion of fructose 6-phosphate (F6P) and mannose-6-phosphate;
ii) manB which encodes the protein phosphomannomutase (EC 5.4.2.8, UniProt accession nr P24175), which is involved in the biosynthesis of GDP-mannose by catalyzing conversion mannose-6-phosphate into mannose-1-phosphate;
iii) manC which encodes the protein mannose-1-phosphate guanylyltransferase guanylyltransferase (EC: 2.7.7.13, UniProt accession nr P24174), which is involved in the biosynthesis of GDP-mannose through synthesis of GDP-mannose from GTP and α-D-mannose-1-phosphate;
iv) gmd which encodes the protein GDP-mannose-4,6-dehydratase (UniProt accession nr POAC88), which catalyzes the conversion of GDP-mannose to GDP-4-dehydro-6-deoxy-D-mannose;
v) wcaG (fcl) which encodes the protein GDP-L-fucose synthase (EC 1.1.1.271, UniProt accession nr P32055) which catalyses the two-step NADP-dependent conversion of GDP-4-dehydro-6-deoxy-D-mannose to GDP-fucose.

Accordingly, it is preferred that the genetically engineered cell, when producing one or more fucosylated heterologous products, overexpresses either the entire colonic acid gene cluster and/or one or more genes of the de novo GDP-fucose pathway selected from the group consisting of manA, manB, manC, gmd and wcaG.

In one or more exemplary embodiment(s), the colanic acid gene cluster responsible for the formation of GDP-fucose may be expressed from its native genomic locus. The expression may be actively modulated to increase GDP-fucose formation. The expression can be modulated by swapping the native promoter with a promoter of interest, and/or increasing the copy number of the colanic acid genes coding said protein(s) by expressing the gene cluster from another genomic locus than the native, or episomally expressing the colanic acid gene cluster or specific genes thereof.

In relation to the present disclosure, the term “native genomic locus”, in relation to the colanic acid gene cluster, relates to the original and natural position of the gene cluster in the genome of the genetically engineered cell.

a Method for Producing N-Acetylated- and/or Sialylated Molecules

The present disclosure also relates to a method for producing one or more N-acetylated- and/or sialylated molecules comprising the steps of:
a) Providing a genetically modified cell according to the disclosure; and
b) Culturing the cell according to (a) in a suitable cell culture medium to produce said N-acetylated- and/or sialylated molecule.

Preferably the N-acetylated- and/or sialylated molecule is selected from the group of oligosaccharides, glycoproteins and glycolipids. If the molecule is N-acetylated then, preferably, at least one N-acetylation in the molecule is a N-acetylglucosamine moiety.

One embodiment of the present disclosure relates to a method for producing one or more oligosaccharides, such as human milk oligosaccharides (HMO), comprising the steps of:
a) Providing a genetically modified cell according to the disclosure; and
b) Culturing the cell according to (a) in a suitable cell culture medium to produce said HMO.

The embodiments mentioned above for the genetically modified cell according to the disclosure also apply to the method according to the disclosure.

In embodiments the one and more oligosaccharide is selected from the group consisting of: LNT-II, LNT, LNnT, LNH, LNnH, pLNH, pLNnH, LNFP-I, LNFP-II, LNFP-III, LNFP-V, LNFP-V, LNFP-VI, LNDFH-I, LNDFH-II, LNDFH-III, F-pLNnH, DF-LNH a, DF-LNH b, DF-LNH c, DF-pLNnH, TF-pLNnH, FDS-LNH, TF-LNH, DS-LNH, F-LNH-I, F-LNH-II, F-LNH-III, FLST-a (S-LNFP-II), FLST-b, pLNnH, FLST-c, FLST-d, FSL, sialyl-Lewis-X, 3′SL, 6′SL, LST-a, LST-b, LST-c, LST-d, DSLNT, SLNH, SLNH-II, and any mixture thereof or polysaccharides selected from the group consisting of chitin, chitosan, hyaluronic acid, chondroitin sulfate or heparan sulfate.

In a particular embodiment, the human milk oligosaccharide that is produced is selected from the group consisting of: LNT, LNT-II, LNnT, LNH, LNnH, pLNH, pLNnH, LNFP-I, LNFP-II, LNFP-III, LNFP-V, LNFP-VI, LNDFH-I, LNDFH-II, LNDFH-III, F-pLNH, pLNnH, FLST-a, FLST-b, FLST-c, FSL, 3′SL, 6′SL, LST-a, LST-b, LST-c, DSLNT, SLNH, SLNH-II, DF-LNH a, DF-LNH b, DF-LNH c, DF-pLNnH, TF-pLNnH, pLNnH, FDS-LNH, TF-LNH, DS-LNH, F-LNH-I, F-LNH-II, F-LNH-III and any mixture thereof. Preferably, the human milk oligosaccharide to be produced is a mixture of LNT-II, LNnT, and pLNnH, or a mixture of LNT-II, LNT, and pLNH2 or a sialylated human milk oligosaccharide, such as 3′SL or 6′SL.

In an embodiment, the culturing step b) is performed in the presence of an energy source (carbon source) selected from the group consisting of glucose, sucrose, fructose, xylose and glycerol. Preferably, the carbon source is one that results in overflow metabolism when present in excess amounts during fermentation. Preferably, the carbon source is glucose or sucrose, including hydrolyzed sucrose.

In an embodiment, the cultivation is a fed batch fermentation or a continuous fed-batch (feed and bleed fermentation), where the carbon source is continuously feed to the fermentation broth during the fermentation.

In an embodiment, the method of the present disclosure further comprises providing an acceptor saccharide as substrate for the HMO formation, the acceptor saccharide comprising at least two monosaccharide units, which is exogenously added to the culture medium and/or has been produced by a separate microbial fermentation. In a preferred embodiment the substrate for HMO formation is lactose which is provided (e.g. fed) to the culture during the fermentation of the genetically engineered cell. Alternative acceptor saccharides can for example be LNT-II, 2′FL, 3FL, 3′SL, 6′SL, LNnT and LNT, which can be exogenously added to the culture medium and/or have been produced by a separate microbial fermentation. Preferably, the acceptor saccharide is provided at the end of the batch phase if the fermentation is a fed-batch or continuous fed-batch fermentation.

Culturing or fermenting (used interchangeably herein) in a controlled bioreactor typically comprises (i) a first phase of exponential cell growth in a culture medium ensured by a carbon source, and (ii) a second phase of cell growth in a culture medium run under carbon limitation, where the carbon source is added continuously together with the acceptor oligosaccharide, such as lactose, allowing formation of the HMO product in this phase. By carbon (sugar) limitation is meant the stage in the fermentation where the growth rate is kinetically controlled by the concentration of the carbon source (sugar) in the culture broth, which in turn is determined by the rate of carbon addition (sugar feed-rate) to the fermenter.

With regards to the suitable cell medium used in the fermentation process, there are no limitations. The culture medium may be semi-defined, i.e., containing complex media compounds (e.g., yeast extract, soy peptone, casamino acids, etc.), or it may be chemically defined, without any complex compounds. The carbon source can be selected from the group consisting of glucose, sucrose, fructose, xylose and glycerol. In one or more exemplary embodiments, the culturing media is supplemented with one or more energy and carbon sources selected form the group containing glycerol, sucrose and glucose. Preferably, the carbon source is glucose or sucrose.

In one or more exemplary embodiments, the culturing media contains sucrose as the sole carbon and energy source. In one or more exemplary embodiments, the genetically engineered cell comprises one or more heterologous nucleic acid sequence encoding one or more heterologous polypeptide(s) which enables utilization of sucrose as sole carbon and energy source of said genetically engineered cell, such as a PTS-dependent sucrose utilization system, further comprising the scrYA and scrBR operons as described in WO2015/197082.

In an embodiment, the HMO is recovered from the cultivation broth and/or the biomass, preferably at the end of the culturing/fermentation process. Suitable methods for recovering the produced HMO are known to a skilled person, and for example described in WO2016095924, WO2015188834, WO2017152918, WO2017182965, or US20190119314.

### Use of a Genetically Modified Cell

The present disclosure also relates to any commercial use of the genetically modified cell(s) disclosed herein, such as, but not limited to, in a method for producing one or more human milk oligosaccharide (HMO).

In an embodiment of the present disclosure, the genetically modified cell and/or the nucleic acid construct according to the disclosure is used in the manufacturing of HMOs. Preferably, the genetically modified cell is used in large scale manufacturing of one or more HMOs. In one embodiment of the disclosure a large-scale fermentation reaches a final fermentation volume above 1,000 L, such as above 10,000 L, such as above 50,000 L, preferably above 100,000 L.

N-Acetylated- and/or Sialylated Molecules Produced by the Method According to the Disclosure

Another aspect of the present disclosure relates to N-acetylated- and/or sialylated molecules produced by the method according to the disclosure.

In embodiments the one or more N-acetylated- and/or sialylated molecules selected from the group of oligosaccharides, glycoproteins and glycolipids. If the molecule is N-acetylated then, preferably, at least one N-acetylation in the molecule is a N-acetylglucosamine moiety.

In preferred embodiments the N-acetylated- and/or sialylated molecule is an oligosaccharide, such as a human milk oligosaccharides (HMO).

The N-acetylated- and/or sialylated molecule, such as an HMO, thus produced may be in the form of a powder, a composition, a suspension, or a gel.

### Sequences

The current disclosure contains a sequence listing in text format and electronical format, which is hereby incorporated by reference.

The table below is a summary of sequences listed in the present disclosure.

| | | | | Genome location of DNA |
|---|---|---|---|---|
| SEQ | | | | in Escherichia coli K-12 |
| ID | | | | chromosome, complete |
| NO | Name | type | Organism | genome (CP047127.1) |
| 1 | GlmM | AA | Esherichia coli | 2429550 to 2430887 |
| 2 | GlmU | AA | Esherichia coli | 1838455 to 1839825 |
| 3 | GlmS | AA | Esherichia coli | 1839987 to 1841816 |
| 4 | RapZ | AA | Esherichia coli | 2405651 to 2406505 |
| 5 | yhbJ | DNA | Esherichia coli | 2405651 to 2406505 |
| 6 | IgtA | AA | Neisseria | |
| | | | meningitidis | |
| 7 | GalTK | AA | Helicobacter | |
| | | | pylori | |
| 8 | GalT | AA | Helicobacter | |
| | | | pylori | |
| 9 | Poral | AA | Pasteurella | |
| 10-34 | Promoters | DNA | oralis | |
| 35 | neuBCA | DNA | Campylobacter | |
| | | | jejuni | |

### EXAMPLES

### Methods

### Strain Engineering

The strains (genetically engineered cells) constructed in the present disclosure were based on Escherichia coli K-12 DH1 with the genotype: F⁻, λ⁻, gyrA96, recA1, relA1, endA1, thi-1, hsdR17, supE44. Additional modifications were made to the E. coli K-12 DH1 strain to generate the MDO mother/platform strain with the following modifications: lacZ: deletion of 1.5 kbp, lacA: deletion of 0.5 kbp, nanKETA: deletion of 3.3 kbp, melA: deletion of 0.9 kbp, wcaJ: deletion of 0.5 kbp, mdoH: deletion of 0.5 kbp, and insertion of Plac promoter upstream of the gmd gene. Unless stated otherwise, standard techniques, vectors, control sequence elements, and other expression system elements known in the field of molecular biology were used for nucleic acid manipulation, transformation, and expression. Such standard techniques, vectors, and elements can be found, e.g., in: Ausubel et al. (eds.), Current Protocols in Molecular Biology (1995) (John Wiley & Sons); Sambrook, Fritsch, & Maniatis (eds.), Molecular Cloning (1989) (Cold Spring Harbor Laboratory Press, NY); Berger & Kimmel, Methods in Enzymology 152: Guide to Molecular Cloning Techniques (1987) (Academic Press); Bukhari et al. (eds.), DNA Insertion Elements, Plasmids and Episomes (1977) (Cold Spring Harbor Laboratory Press, NY); Miller, J. H. Experiments in molecular genetics (1972) (Cold spring Harbor Laboratory Press, NY).

### Deep Well Assay

For strain characterization, the following deep well assay protocol has been applied:

### Determination of UDP-GlcNAc Levels in Strain Variants of the MDO Mother Strain

The strains used in the following examples were screened in 96 deep well plates using a 4-day protocol. During the first 24 hours, precultures were grown to high densities and subsequently transferred to a medium that allowed induction of gene expression and UDP-GlcNAc formation. More specifically, during day 1, fresh precultures were prepared using a basal minimal medium supplemented with magnesium sulphate, thiamine and glucose. The precultures were incubated for 24 hours at 34° C. and 1000 rpm shaking and then further transferred to a new basal minimal medium (BMM, pH 7.5) in order to start the main culture. The new BMM was supplemented with magnesium sulphate, thiamine, a bolus of 20% glucose solution (0.1-0.15 g/L) and 50% sucrose solution (40-45 g/L), which was provided as carbon source, accompanied by the addition of sucrose hydrolase (invertase), so that glucose was released at a rate suitable for C-limited growth. The main cultures were incubated for 72 hours at 28° C. and 1000 rpm shaking.

### Determination of HMO Levels Produced by the Strains

The strains used in the following examples were screened in 96 deep well plates using a 4-day protocol. During the first 24 hours, precultures were grown to high densities and subsequently transferred to a medium that allowed induction of gene expression and product formation. More specifically, during day 1, fresh precultures were prepared using a basal minimal medium supplemented with magnesium sulphate, thiamine and glucose. The precultures were incubated for 24 hours at 34° C. and 1000 rpm shaking and then further transferred to a new basal minimal medium (BMM, pH 7.5) in order to start the main culture. The new BMM was supplemented with magnesium sulphate, thiamine, a bolus of 20% glucose solution (0.1-0.15 g/L) and a bolus of lactose solution (5-20 g/L). Moreover, 50% sucrose solution (40-45 g/L) was provided as carbon source, accompanied by the addition of sucrose hydrolase (invertase), so that glucose was released at a rate suitable for C-limited growth. The main cultures were incubated for 72 hours at 28° C. and 1000 rpm shaking.

### Example 1—Genetic Engineering Tools to Increase the UDP-GlcNAc Pool in E. coli Strains

Based on the platform strain (MDO), various further modifications were made to assess their potential effect on the UDP-GlcNAc pool in the cell as they are all related to the metabolic pathway for producing UDP-GlcNAc (FIG. 1). The modifications are summarized in Table 10 below to obtain the fully chromosomal strains MP1, MP2, MP3, MP4, MP5, MP6, MP7, MP8, MP9, MP10, and MP11.

TABLE 10
Genotypes of the strains bearing modifications of genes involved
in the biosynthesis or the regulation of the UDP-GlcNAc pathway

| Strain | |
|---|---|
| ID | Genotype |
| MDO/ | F-λ- ΔendA1 ΔrecA1 ΔrelA1 ΔgyrA96 Δthi-1 glnV44 |
| Control | hsdR17(rK-mK-) ΔlacZ wcaF::Plac ΔnanKETA ΔlacA ΔmelA |
| | ΔwcaJ ΔmdoH |
| MP1 | MDO, ΔyhbJ |
| MP2 | MDO, ΔnagB |
| MP3 | MDO, PglpF-glmS |
| MP4 | MDO, PglpF-glmU |
| MP5 | MDO, PglpF-glmM |
| MP6 | MDO, PglpF-glmS, PglpF-glmU, PglpF-glmM |
| MP7 | MDO, ΔyhbJ, PglpF-glmS |
| MP8 | MDO, ΔyhbJ, PglpF-glmU |
| MP9 | MDO, ΔyhbJ, PglpF-glmM |
| MP10 | MDO, ΔyhbJ, PglpF-glmM, PglpF-glmU |
| MP11 | MDO, ΔyhbJ, PglpF-glmM, PglpF-glmU, PglpF-glmS |

From Table 10, it can be observed that the strains bear a deletion of the genes yhbJ or nagB (strains MP1 and MP2), whose protein products are closely associated with the UDP-GlcNAc pathway, or bear an extra PgIpF-driven copy of one or all of the key biosynthetic genes glmM, glmU, glmS (strains MP3 to MP6), or combinations of the aforementioned modifications (strains MP7 to MP11).

The strains in Table 10 were screened using the UDP-GlcNAc Deep Well Assay described in the methods section as replicates of two and the UDP-GlcNAc levels were assessed using HPLC and the identity of the UDP-GlcNAc HPLC-peak was confirmed by LC-MS.

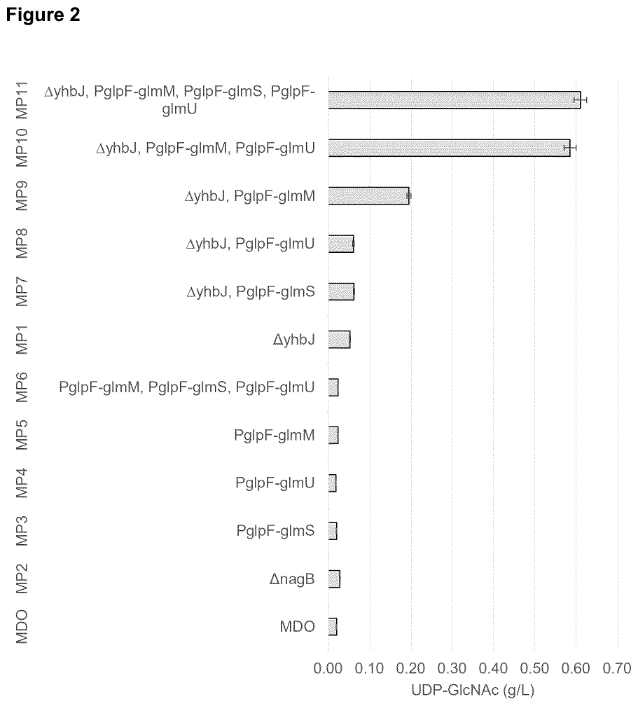

The results are shown in Table 11 and FIG. 2, respectively. In Table 11 and FIG. 2, respectively, the measured concentrations [g/L] of UDP-GlcNAc are given for all strains that are listed in Table 10.

TABLE 11
Measured concentrations [g/L] of UDP-
GlcNAc of all strains listed in Table 10

| | | UDP- | |
|---|---|---|---|
| | | GlcNAc | |
| Strain | Mutations | [g/L] | OD |
| MDO | control | 0.02 | 11.55 |
| MP2 | ΔnagB | 0.03 | 12.40 |
| MP3 | PglpF-glmS | 0.02 | 11.90 |
| MP4 | PglpF-glmU | 0.02 | 12.35 |
| MP5 | PglpF-glmM | 0.02 | 11.35 |
| MP6 | PglpF-glmS + PglpF-glmU + | 0.02 | 9.70 |
| | PglpF-glmM | | |
| MP1 | Δyhbj | 0.05 | 11.85 |
| MP7 | Δyhbj + PglpF-glmS | 0.06 | 9.00 |
| MP8 | Δyhbj + PglpF-glmU | 0.06 | 12.75 |
| MP9 | Δyhbj + PglpF-glmM | 0.20 | 8.40 |
| MP10 | Δyhbj + PglpF-glmM + | 0.59 | 9.35 |
| | PglpF-glmU | | |
| MP11 | Δyhbj PglpF-glmS + | 0.61 | 10.05 |
| | PglpF-glmU + PglpF-glmM | | |

From Table 11 and FIG. 2, it can be observed that the deletion of the yhbJ gene (MP1) led to an increase in the UDP-GlcNAc concentration as compared to the control strain (MDO). It can also be observed that said increase was more pronounced as compared to e.g., the deletion of the nagB gene (MP2) that is also involved in the biosynthesis of UDP-GlcNAc.

It can also be observed that despite the fact that the introduction of a PgIpF-driven copy of glmU or glmS alone did not lead to an increase in UDP-GlcNAc concentration (see strains MP3 and MP4, respectively) the introduction of a PgIpF-driven copy of glmU or glmS, respectively, in combination with the deletion of the yhbJ gene (see strain MP8/MP7) led to a slight increase in the UDP-GlcNAc concentration compared to the deletion of the yhbJ gene alone (MP1). The effect is thus more than additive.

With respect to a PgIpF-driven copy of glmM alone, this also did not lead to an increase in UDP-GlcNAc concentration (see strain MP5), the combination of the deletion of the yhbJ gene and the introduction of a PgIpF-driven copy of glmM (see strain MP9), however, led to an increase in UDP-GlcNAc concentration that was beyond mere additive effects. Hence, a synergistic effect of said modifications is indicated.

It can further be observed that the introduction of PgIpF-driven copies of glmU together with glmM and the deletion of the yhbJ gene (see strain MP10) led to an increase in the concentration of UDP-GlcNAc that was almost 3-fold above what was achieved with the already synergistic combination of glmM overexpression with the yhbJ deletion (MP9).

Similarly, it can be observed that the introduction of PgIpF-driven copies of glmU, glmS, and glmM combined with the deletion of the yhbJ gene (see strain MP11) led to an increase in the concentration of UDP-GlcNAc that was beyond of what could have been expected based on mere additive effects based on the individual modifications (see strains MP1, MP5, MP3, and MP4) as well as the combination of the PgIpF-driven copies of glmU, glmS and glmM (without deletion of the yhbJ gene, MP6), which did not result in an improvement over the individual modifications (strain MP6 as compared to MP5/MP4/MP3).

Said example shows that the deletion of the yhbJ gene alone or in combination with the overexpression of one or more of glmU, glmS and/or glmM is an efficient tool to increase the concentration of UDP-GlcNAc in E. coli strains. In particular, the combination with overexpression of glmM was found to be highly beneficial.

### Example 2—LNnT Producing E. coli Strains

Since UDP-GlcNAc is used as the donor substrate in the formation of LNT-II, which is further used as acceptor in the formation of LNnT, the influence of an increased UDP-GlcNAc pool on the formation of LNnT has been assessed.

Based on the platform strain (MDO), further modifications were made as summarized in Table 12 below, to obtain the fully chromosomal LNnT strains MP12, MP13, MP14 and MP15. Each of these strains comprises one or two PgIpF-driven copies of a beta-1,3-N-acetyloglucosamine transferase, LgtA gene, from Neisseria meningitidis (SEQ ID NO: 6) and a single PgIpF-driven copy of a beta-1,4-galactosyltransferase, GalT) gene, from Helicobacter pylori 26695 (SEQ ID NO: 8). The only difference between these two strain pairs is (a) the deletion of the yhbJ gene for the strain pair MP12-MP13 and (b) the deletion of the yhbJ gene and the PgIpF-driven overexpression of the genes glmM and glmU for the strain pair MP14-MP15. All four strains can produce the tetrasaccharide HMO LNnT as the main product. Apart from LNnT, LNT-II and pLNnH can also be formed.

TABLE 12
Genotypes of the strains expressing glycosyltransferases (genomically
integrated) that enable LNnT biosynthesis, with and without
the modification of genes encoding proteins associated with the
UDP-GlcNAc biosynthetic pathway in E. coli.

| Strain ID | Genotype |
|---|---|
| MP12 | MDO, x2 PglpF-IgtA, x1 PglpF-galT |
| MP13 | MDO, x2 PglpF-IgtA, x1 PglpF-galT, ΔyhbJ |
| MP14 | MDO, x1 PglpF-IgtA, x1 PglpF-galT |
| MP15 | MDO, x1 PglpF-IgtA, x1 PglpF-galT, |
| | ΔyhbJ, x1 PglpF-glmM, x1 PglpF-glmU |

The strains in Table 12 were screened using the HMO Deep Well Assay as described in the methods section as replicates of four and the levels of all HMOs produced by the strains were assessed using HPLC.

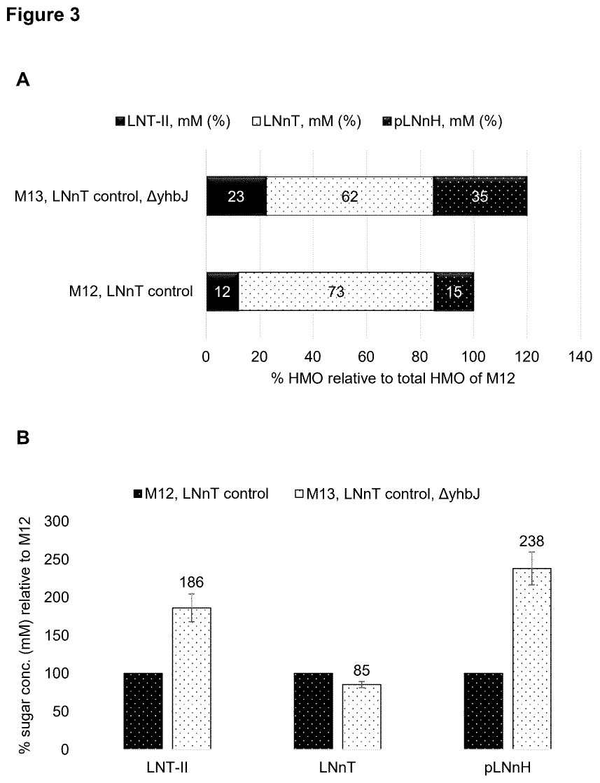

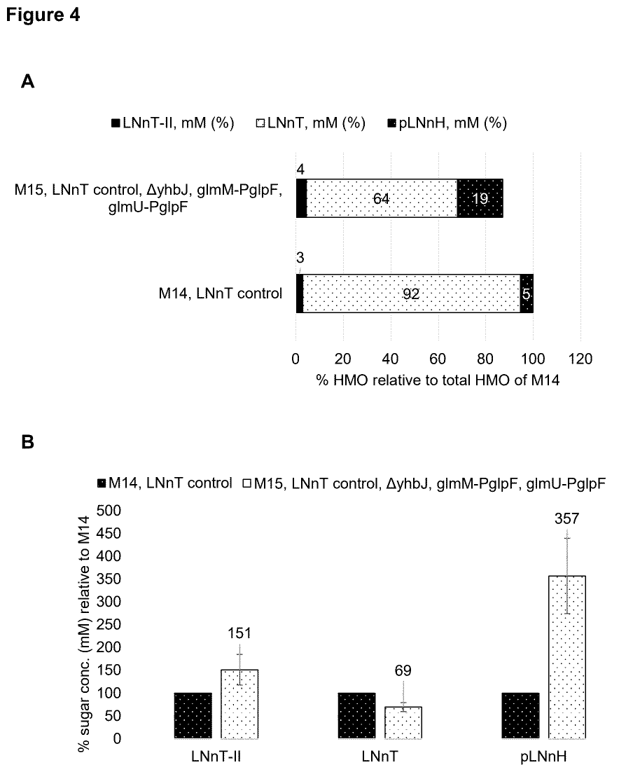

The results for the 2 strain pairs are shown in FIGS. 3 (MP12-MP13) and 4 (MP14-MP15). In FIGS. 3A and 4A, the amount of HMO produced (mM) by each test strain (MP13 and MP15) is shown relative to the total amount of HMO produced in the control strain (MP12 or MP14, respectively). FIGS. 3B and 4B show the relative concentrations (mM) of each individual oligosaccharide (sugar) relative to the amounts of the same individual oligosaccharide (sugar) produced by the control strain (MP12 or MP14, respectively) set to 100%.

From FIG. 3A it can be observed that the deletion of the yhbJ gene led to an increase in the total HMO content of 20%, while the combination of 3 modifications (i.e., deletion of the yhbJ gene and overexpression of the glmU and glmM genes) in strain MP15 led to a slight decrease of approximately 13% in total HMO formation compared to the control strain MP14 (FIG. 4A).

Moreover, from FIG. 3B it can be observed that the deletion of the yhbJ gene led to a slight decrease of the amounts of the main product LNnT in favor of the HMOs LNT-II and pLNnH. This shows that the deletion of the yhbJ gene allows for an increase in the proportions of the minor HMO products LNT-II and pLNnH at the expense of the major product LNnT. This is surprising and shows that not only the total oligosaccharide content can be influenced by the deletion of the yhbJ gene, but also the individual proportions of the produced HMOs, in particular if production of the hexa-oligosaccharide pLNnH is desired deletion of yhbJ appear to be favorable.

Although the total HMO content of the strain MP15 that bears an extra genomic copy of the glmU and glmM genes along with a deletion of the yhbJ gene was slightly decreased compared to the control strain MP14 or even the modified strain MP13, the previously observed ΔyhbJ-mediated increase in the proportions of the minor HMO product pLNnH (FIG. 3B), was markedly amplified with the additional overexpression of the glmM and glmU genes (FIG. 4B). Specifically, the amount of pLNnH formed by the strain MP15 was more than tripled compared to the control strain MP14 and much higher than the pLNnH concentrations observed for the strain MP13 (FIG. 4B).

The individual proportions of the formed HMOs are also shown in FIGS. 3A and 4A. In FIG. 3A, it can be seen that the deletion of the yhbJ gene led to a relative decrease of the proportion of the main product (LNnT) from 73% to 62%. Moreover, a relative increase of LNT-II from 12% to 23% could be observed. Further, a relative increase of pLNnH from 15% to 35% could be observed. Similarly, in FIG. 4A, we observed that the combined deletion of the yhbJ gene and overexpression of the glmU and glmM genes led to a relative decrease of the proportion of the main product (LNnT) from 92% to 64%, a relative increase of LNT-II from 3% to 4% and finally a relative increase of pLNnH from 5% to 19%.

These results demonstrate that the deletion of the yhbJ gene alone or when combined with the overexpression of the glmU and glmM genes allows for a shift of the proportions of the individual HMOs in the mixture of HMO produced by the strain, with the shift being stronger for a strain bearing all 3 modifications.

### Example 3—LNT Producing E. coli Strains

Based on the platform strain (MDO), further modifications were made as summarized in Table 13 below, to obtain the fully chromosomal LNT producing strains MP16-MP19. Each of these strains comprise one or two PgIpF-driven copies of a beta-1,3-N-acetyloglucosamine transferase, IgtA gene, from Neisseria meningitidis (SEQ ID NO: 6) (strain pairs MP16-MP17 or MP18-MP19, respectively) and a single PgIpF-driven copy of a beta-1,3-galactosyltransferase, galTK gene, from Helicobacter pylori 43504 (SEQ ID NO: 7). The only difference between these two strain pairs is (a) the deletion of the yhbJ gene for the strain pair MP16-MP17 and (b) the deletion of the yhbJ gene and the PgIpF-driven overexpression of the genes glmM and glmU for the strain pair MP18-MP19. All four strains can produce the tetrasaccharide HMO LNT as the main product. Apart from LNT, LNT-II and pLNH2 can also be formed.

TABLE 13
Genotypes of the strains expressing glycosyltransferases
that enable LNT biosynthesis from genomic copies of the
glycosyltransferases, with and without the simultaneous
modification of genes encoding proteins associated with
the UDP-GlcNAc biosynthetic pathway in E. coli.

| Strain ID | Genotype |
|---|---|
| MP16 | MDO, x2 PglpF-IgtA, x1 PglpF-galTK |
| MP17 | MDO, x2 PglpF-IgtA, x1 PglpF-galTK, ΔyhbJ |
| MP18 | MDO, x1 PglpF-IgtA, x1 PglpF-galTK |
| MP19 | MDO, x1 PglpF-IgtA, x1 PglpF-galTK, |
| | ΔyhbJ, x1 PglpF-glmM,, x1 PglpF-glmU |

The strains in Table 13 were screened using the HMO Deep Well Assay as described in the methods section as replicates of four and the levels of all HMOs produced by the strains were assessed using HPLC.

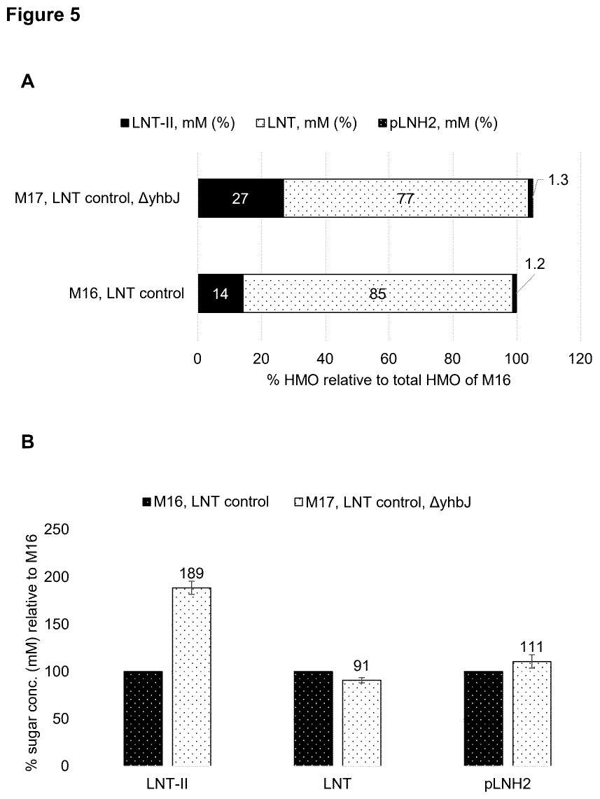

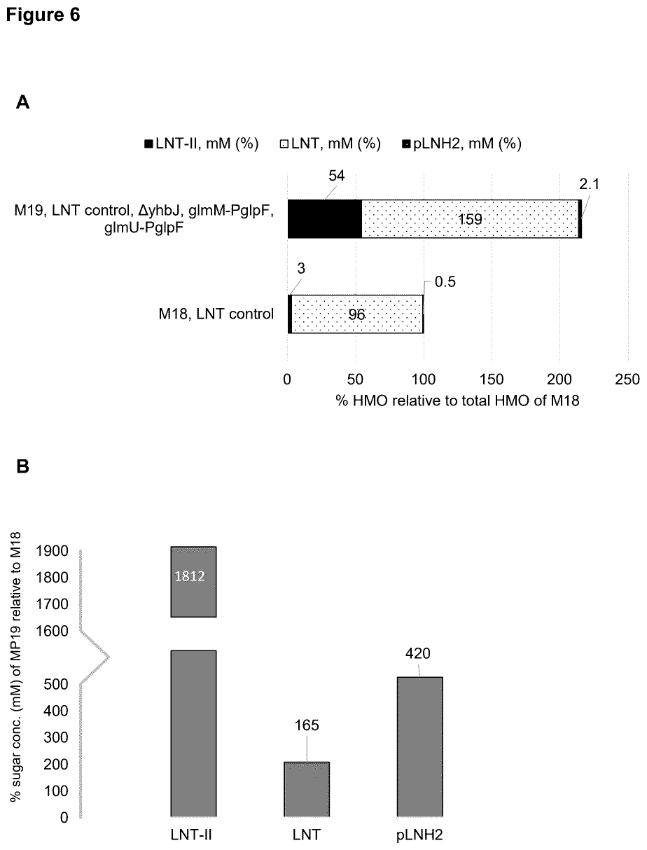

The results for the 2 strain pairs are shown in FIGS. 5 (MP16-MP17) and 6 (MP18-MP19). In FIGS. 5A and 6A, the amount of HMO produced (mM) by each test strain (M17 and MP19) is shown relative to the total amount of HMO produced in the control strain (MP16 or MP18, respectively). FIGS. 5B and 6B show the relative concentrations (mM) of each individual oligosaccharide (sugar) relative to the amounts of the same individual oligosaccharide (sugar) produced by the control strain (MP16 or MP18, respectively) set to 100%. FIG. 6B is not displaying the oligosaccharide levels of the control strains since these are pr. default set to 100%.

From FIG. 5A it can be observed that the deletion of the yhbJ gene led to only a minor increase in the total HMO content of approximately 5%, while the combination of 3 modifications (i.e., deletion of the yhbJ gene and overexpression of the glmU and glmM genes) in strain MP19 led to more than double total HMO formation compared to the control strain MP18 (FIG. 6A).

Moreover, from FIG. 5B it can be observed that the deletion of the yhbJ gene led to a slight decrease of the amounts of the main product LNT in favor of a slight increase in the by-product pLNH2 and a significant increase of the HMO LNT-II, whose concentration was almost doubled in the acquired final HMO profile. This shows that the deletion of the yhbJ gene allows for an increase in the proportions of the minor HMO product LNT-II, which is accompanied by a minor compromise in the final titer of the major product LNT.

As mentioned above, the total HMO content of the strain MP19 that bears an extra genomic copy of the glmU and glmM genes along with a deletion of the yhbJ gene was markedly higher than the total HMO content of the control strain MP18 or even the modified strain MP17 (FIG. 6A). Contrary to the remarkable AyhbJ-mediated increase in the proportion of the minor HMO product LNT-II alone (FIG. 5B), the additional overexpression of the glmM and glmU genes led to an extraordinary boost in the concentrations of not only LNT-II but also LNT and pLNH2 (1.5- and 4-fold higher concentrations, respectively) (FIG. 6B).

The individual proportions of the formed HMOs are also shown in FIGS. 5A and 6A. In FIG. 5A, it can be seen that the deletion of the yhbJ gene led to a relative decrease of the proportion of the main product (LNT) from 85% to 77%. Moreover, a relative increase of LNT-II from 14% to 27% could be observed. Similarly, in FIG. 6A, we observed that the combined deletion of the yhbJ gene and overexpression of the glmU and glmM genes led to a relative increase of the proportion of the main product (LNT) from 96% to 159%, a relative increase of LNT-II from 3% to 54% and finally a relative increase of pLNH2 from 0.5% to 2.1%.

These results demonstrate that the deletion of the yhbJ gene alone or when combined with the overexpression of the glmU and glmM genes allows for a shift of the proportions of the individual HMOs in the mixture of HMO produced by the strain, with the shift being stronger for a strain bearing all 3 modifications. This is surprising and shows that not only the total sugar content can be influenced by modifying these 3 genes in the manner suggested in the present disclosure, but also the individual proportions of the produced HMOs.

### Example 4—3′SL Producing E. coli Strains

The formation of 3′SL uses CMP-Neu5Ac as donor for the reaction catalyzed by a suitable alpha-2,3-sialyltranferase. In the E. coli cells used in the present example, CMP-Neu5Ac is formed from UDP-GlcNAc by inserting the genes of the sialic acid sugar nucleotide pathway comprising exogenous UDP-GlcNAc 2-epimerase (neuC), a Neu5Ac synthase (neuB), and a CMP-Neu5Ac synthetase (neuA).

Based on the platform strain (MDO), further modifications were made as summarized in Table 14 below, to obtain the strain MP20 as control strain that can produce the HMO 3′SL. Further, the test strains MP21-MP23 have been prepared that only differed from MP20 in that the yhbJ gene alone was deleted from the chromosome of the MP20 strain (to create the strain MP21) or the yhbJ gene deletion was combined with the overexpression of the glmS gene (to create the strain MP22) or the yhbJ gene deletion was combined with the overexpression of the glmM gene (to create the strain MP23).

All four strains express a genomically integrated alpha-2,3-sialyltransferase, Poral gene, which encodes the wild-type enzyme of SEQ ID NO: 9 from Pasteurella oralis. Furthermore, the strain contains a high-copy plasmid, pBS-neuBCA-amp, bearing the neuBCA operon from Campylobacter jejuni (SEQ ID NO: 35).

TABLE 14
Genotypes of the strains bearing modifications of genes involved
in the biosynthesis of 3′SL, with and without the simultaneous
modification of genes encoding proteins associated with the
UDP-GlcNAc biosynthetic pathway in E. coli.

| Strain ID | Genotype |
|---|---|
| MP20 | MDO, PglpF-Poral, pBS-neuBCA(Plac) |
| MP21 | MDO, PglpF-Poral, pBS-neuBCA(Plac), ΔyhbJ |
| MP22 | MDO, PglpF-Poral, pBS-neuBCA(Plac), |
| | ΔyhbJ, x1 PglpF-glmS |
| MP23 | MDO, PglpF-Poral, pBS-neuBCA(Plac), |
| | ΔyhbJ, x1 PglpF-glmU |

The strains in Table 14 were screened using the HMO Deep Well Assay as described in the methods section as replicates of four and the 3′SL levels were assessed using HPLC.

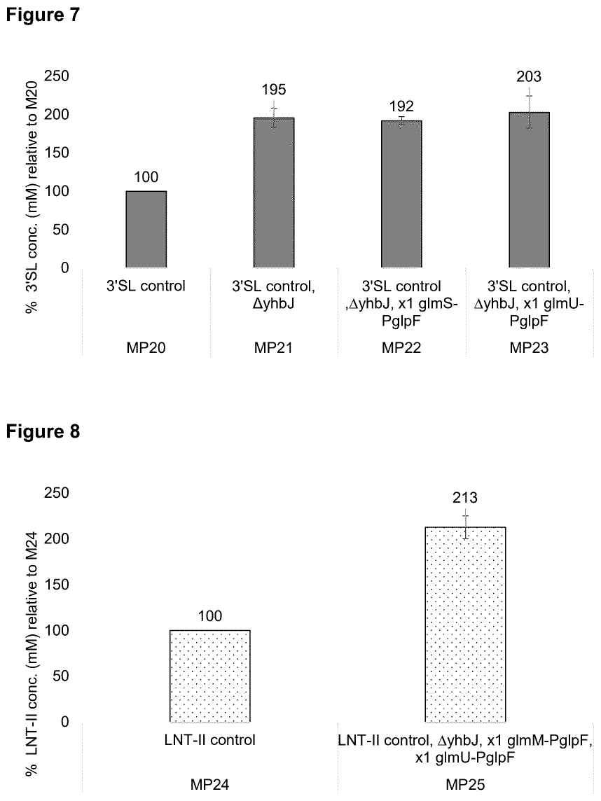

The results are shown in FIG. 7 as relative concentrations (mM) of 3′SL, wherein the amount of 3′SL found in the control strain MP20 (no deletion of the yhbJ gene or overexpression of glmU and/or glmS genes) has been set to 100%.

It can be observed from FIG. 7 that the deletion of the yhbJ gene led to a marked increase in the concentration of 3′SL by a factor of approximately 2 (strain MP21 vs. strain MP20). Slightly better results were obtained when the deletion of the yhbJ gene was accompanied by the overexpression of the glmU gene (strain MP23 vs. strain MP21), whereas the deletion of the yhbJ gene was accompanied by the overexpression of glmS did not appear to have any impact beyond what could be observed with the yhbJ deletion alone in the HMO deep well assay used.

The above-mentioned observations demonstrate that the deletion of the yhbJ gene alone or when combined particularly with the overexpression of the glmU gene, can significantly enhance the production yields of 3′SL in an E. coli strain that can produce 3′SL.

### Example 5—LNT-II Producing E. coli Strains

The formation of LNT-II uses UDP-GlcNAc as donor for the reaction catalyzed by a suitable beta-1,3-N-acetyloglucosamine. In the E. coli cells used in the present example, UDP-GlcNAc is formed in the innate biosynthetic pathway of E. coli cells involving the genes, glmM, glmS and glmU.

Based on the platform strain (MDO), further modifications were made as summarized in Table 15 below, to obtain the strain MP24 as control strain that can produce the HMO LNT-II. Further, the test strain MP25 has been prepared that only differed from MP24 in that the yhbJ gene was deleted from the chromosome of the MP24 strain and the glmM and glmU genes were overexpressed. Both MP24 and MP25 bear a single, PgIpF-driven genomic copy of a beta-1,3-N-acetyloglucosamine transferase, IgtA gene, from Neisseria meningitidis (SEQ ID NO: 6), while the MP25 strain also bears extra genomic copies of the glmU and glmM genes under the control of the PgIpF promoter.

TABLE 15
Genotypes of the strains bearing modifications of genes involved
in the biosynthesis of LNT-II, with and without the simultaneous
modification of genes encoding proteins associated with the
UDP-GlcNAc biosynthetic pathway in E. coli.

| Strain ID | Genotype |
|---|---|
| MP24 | MDO, x1 PglpF-IgtA |
| MP25 | MDO, x1 PglpF-IgtA, ΔyhbJ, x1 |
| | PglpF-glmM, x1 PglpF-glmU |

The strains in Table 15 were screened using the HMO Deep Well Assay as described in the methods section as replicates of four and the LNT-II levels were assessed using HPLC.

The results are shown in FIG. 8 as relative concentrations (mM) of LNT-II, wherein the amount of LNT-II found in the control strain MP24 (no deletion of the yhbJ gene or overexpression of glmU and glmS genes) has been set to 100%.

It can be observed from FIG. 8 that the combined deletion of the yhbJ gene and the overexpression of the glmM and glmU genes more than doubled the concentration of LNT-II that was present in the acquired final HMO profile.

The above-mentioned observations demonstrate that the combined deletion of the yhbJ gene and the overexpression of the glmM and glmU genes can significantly enhance the production yields of LNT-II in an E. coli strain that can produce LNT-II, which correlates very well with the data observed in example 1, where the UDP-GlcNAc pool is significantly increased in a MDO strain (not expressing HMO) with this particular modification.

1. A genetically modified cell capable of producing UDP-N-acetylglucosamine (UDP-GlcNAc), wherein the mRNA stability of the native glucosamine-6-phopshate synthase (glmS) is enhanced by modifying the cell such that it has decreased or total loss of function of RNase adapter protein RapZ (yhbJ).

2. The genetically modified cell of to claim 1, wherein the gene encoding the RNase adapter protein RapZ (yhbJ) is deleted or rendered dysfunctional.

3. The genetically modified cell of claim 1, wherein the cell is modified further to overexpress phosphoglucosamine mutase (GlmM).

4. The genetically modified cell of claim 1, wherein the cell further comprises at least one of the following modifications:
a) overexpression of N-acetylglucosamine-1-phosphate uridyltransferase (GlmU), and/or
b) overexpression of glucosamine-6-phopshate synthase (GlmS).

5. The genetically modified cell of claim 3, wherein the overexpression is obtained by
a) replacing the native promoter of the gene encoding
i. phosphoglucosamine mutase (glmM), and/or
ii. N-acetylglucosamine-1-phosphate uridyltransferase (glmU), and/or
iii. glucosamine-6-phopshate synthase (glmS),
with a stronger promoter; and/or
b) inserting a recombinant nucleic acid encoding
i. phosphoglucosamine mutase (glmM) comprising the amino acid sequence of SEQ ID NO: 1, or an amino acid sequence with at least 90% sequence identity to SEQ ID NO: 1,
ii. N-acetylglucosamine-1-phosphate uridyltransferase (glmU) comprising the amino acid sequence of SEQ ID NO: 2, or an amino acid sequence with at least 8990% sequence identity to SEQ ID NO: 2,
iii. glucosamine-6-phopshate synthase (glmS) comprising the amino acid sequence of SEQ ID NO: 3 or an amino acid sequence with at least 90% sequence identity to SEQ ID NO: 3,
into the cell.

6. The genetically modified cell of claim 1, wherein the cell is capable of producing one or more N-acetylated- and/or sialylated molecules selected from the group of oligosaccharides, glycoproteins and glycolipids.

7. The genetically modified cell of claim 6, wherein the cell is capable of producing one or more oligosaccharides selected from the group consisting of: LNT-II, LNT, LNnT, LNH, LNnH, pLNH, pLNnH, LNFP-I, LNFP-II, LNFP-III, LNFP-V, LNFP-V, LNFP-VI, LNDFH-I, LNDFH-II, LNDFH-III, F-pLNnH, DF-LNH a, DF-LNH b, DF-LNH c, DF-pLNnH, TF-pLNnH, FDS-LNH, TF-LNH, DS-LNH, F-LNH-I, F-LNH-II, F-LNH-III, FLST-a (S-LNFP-II), FLST-b, pLNnH, FLST-c, FLST-d, FSL, sialyl-Lewis-X, 3′SL, 6′SL, LST-a, LST-b, LST-c, LST-d, DSLNT, SLNH, and SLNH-II, or any mixture thereof, or polysaccharides selected from the group consisting of chitin, chitosan, hyaluronic acid, chondroitin sulfate and heparan sulfate.

8. The genetically modified cell of claim 1, wherein the cell further comprises the following modifications:
a) a recombinant beta-1,3-N-acetyl-glucosaminyltransferase, and
wherein the cell is capable of producing LNT-II.

9. The genetically modified cell of claim 1, wherein the cell further comprises the following modifications:
a) a recombinant beta-1,3-N-acetyl-glucosaminyltransferase, and
b) a recombinant beta-1,4-galactosyltransferase, and
wherein the cell is capable of producing an HMO mixture comprising LNT-II, LNnT, and pLNnH.

10. The genetically modified cell of claim 1, wherein the cell further comprises the following modifications:
a) a recombinant beta-1,3-N-acetyl-glucosaminyltransferase, and
b) a recombinant beta-1,3-galactosyltransferase, and
wherein the cell is capable of producing an HMO mixture comprising LNT-II, LNT, and pLNH2.

11. The genetically modified cell of claim 1, wherein the cell further comprises the following modifications:
a) an alpha-2,3-sialyltransferase or alpha-2,6-sialyltransferase and
b) a biosynthetic pathway for making a sialic acid sugar nucleotide, and
wherein the cell is capable of producing a sialylated human milk oligosaccharide.

12. The genetically modified cell of claim 11, wherein the sialylated human milk oligosaccharide is selected from the group consisting of 3′SL, 6′SL, LST-a, LST-b, LST-c and DS-LST.

13. The genetically modified cell according to claim 1, wherein the modified cell is a microbial cell selected from the group consisting of Escherichia coli, Bacillus subtilis, Lactobacillus lactis, Corynebacterium glutamicum, Campylobacter sp., Yarrowia lipolytica, Pichia pastoris, and Saccharomyces cerevisiae.

14. A method for producing one or more N-acetylated- and/or sialylated molecules comprising the steps of:
a) providing a genetically modified cell according to claim 6; and
b) culturing the cell according to (a) in a suitable cell culture medium to produce N-acetylated- and/or sialylated molecule(s).

15. The method according to claim 14, wherein the molecule is an oligosaccharide selected from the group consisting of: LNT, LNT-II, LNnT, LNH, LNnH, pLNH, pLNnH, LNFP-I, LNFP-II, LNFP-III, LNFP-IV, LNFP-V, LNFP-VI, LNDFH-I, LNDFH-II, LNDFH-III, F-pLNnH, DF-LNH a, DF-LNH b, DF-LNH c, DF-pLNnH, TF-pLNnH, FDS-LNH, TF-LNH, DS-LNH, F-LNH-I, F-LNH-II, F-LNH-III, FLST-a (S-LNFP-II), FLST-b, pLNnH, FLST-c, FLST-d, FSL, sialyl-Lewis X, 3′SL, 6′SL, LST-a, LST-b, LST-c, LST-d, DSLNT, SLNH, and SLNH-II, or any mixture thereof, or polysaccharides selected from the group consisting of chitin, chitosan, hyaluronic acid, chondroitin sulfate and heparan sulfate.

16. The method according to claim 15, wherein the oligosaccharide is
a) a mixture of LNT-II, LNnT, and pLNnH, or
b) a mixture of LNT-II, LNT, and pLNH2, or
c) a sialylated human milk oligosaccharide, such as 3′SL or 6′SL.

17. The method according to claim 14, wherein the N-acetylated- and/or sialylated molecule is recovered from the cultivation broth and/or the biomass.
